(19) United States
(12) Patent Application Publication
(10) Pub. No.: US 2010/0099694 A1
(43) Pub. Date: Apr. 22, 2010

(54) 2-ANILINO-4-HETEROARYL PYRIMIDINE DERIVATIVES, AND PREPARATION THEREOF AS MEDICAMENTS, PHARMACEUTICAL COMPOSITIONS, AND IN PARTICULAR IKK INHIBITORS

(75) Inventors: Didier BABIN, Montigny (FR); Monsif BOUABOULA, Juvignac (FR); Pierre CASELLAS, Castelnau-le-lez (FR); Serge MIGNANI, Chatenay-Malabry (FR); Jean-Flaubert NGUEFACK, Lattes (FR); Jean WAGNON, Montpellier (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/496,107

(22) Filed: Jul. 1, 2009

Related U.S. Application Data
(63) Continuation of application No. PCT/FR2008/000004, filed on Jan. 2, 2008.

(30) Foreign Application Priority Data
Jan. 5, 2007 (FR) 0700066

(51) Int. Cl. A61K 31/506; C07D 401/14; C12N 9/99; A61P 29/00; A61P 3/10; A61P 35/00

(52) U.S. Cl. 514/275; 544/324; 435/184

(57) ABSTRACT

The disclosure relates to a compound of formula (I):

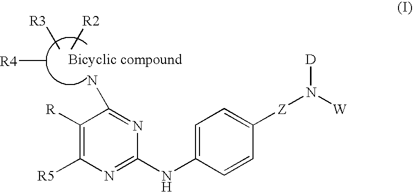

wherein R, R2, R3, R4, R5, z, D and W are as defined in the specification, to compositions containing them, to processes for preparing them, and to their use in the treatment or prevention of conditions capable of being modulated by the inhibition of the activity of protein kinases.

This application is a continuation of International application No. PCT/FR2008/000004, filed Jan. 2, 2008, which is incorporated herein by reference in its entirety; which claims the benefit of priority of French Patent Application No. 0700066, filed Jan. 5, 2007.

The present invention relates to novel 2-anilino-4-heteroaryl-pyrimidine derivatives, to their process of preparation, to the novel intermediates obtained, to their application as medicaments, to the pharmaceutical compositions including them and to the novel use of such 2-anilino-4-heteroaryl-pyrimidine derivatives.

Patent WO200164654-A1 mentions 2,4-di(hetero)arylpyrimidines substituted in the 5 position which are inhibitors of the kinases CDK 2 and FAK; likewise, other aminopyrimidines which are inhibitors of serine-threonine kinases and of CDK are presented in WO2003030909-A1. Patent WO2004046118-A2 describes 2,4-diphenylaminopyrimidine derivatives as inhibitors of cell proliferation.

A series of 5-cyano-2-aminopyrimidines is presented as inhibitors of the kinases KDR and FGFR in WO200078731-A1, of other pyrimidines as inhibitors of FAK and of IGFR in WO2004080980A-1, and also of ZAP-70, FAK and/or Syk tyrosine kinase in WO2003078404-A1, and of the polo-like kinases PLK in WO2004074244-A2, as cytostatic agents.

Likewise, other patents describe pyrimidines which are inhibitors of reverse transcriptase in the treatment of HIV-related infections (WO200185700-A2, WO200185699-A2, WO200027825A1 and WO2003094920A1).

A subject-matter of the present invention is thus novel 2-anilino-4-heteroaryl-pyrimidine derivatives possessing inhibiting effects on protein kinases.

The products of the present invention can thus in particular be used in the prevention or treatment of conditions capable of being regulated by the inhibition of the activity of protein kinases.

Mention is more particularly made, among these protein kinases, of the protein kinase IKK-alpha (IKKα) and IKK-beta (IKKβ).

The compounds of the present invention are kinase inhibitors, in particular inhibitors of IKK-alpha and IKK-beta; consequently, they inhibit NF-KB (nuclear factor kappa B) activity; thus, they can be used in the treatment or prophylaxis of inflammatory diseases, cancer and diabetes.

NF-kB (nuclear factor kappa B) belongs to a family of complexes of transcription factors composed of different combinations of Rel/NF-KB polypeptides. The members of this family of NF-KB-related polypeptides regulate the expression of genes involved in immune and inflammatory responses (Bames P J and Karin M (1997), New Engl. J. Med., 336, 1066-1071, and Baeuerle P A and Baichwal V R (1997), Adv. Immunol., 65, 111-137). Under basal conditions, NF-KB dimers are retained in the inactive form in the cytoplasm by inhibitory proteins which are members of the IKB family (Beg et al., Genes Dev., 7: 2064-2070, 1993; Gilmore and Morin, Trends Genet., 9: 427-433), 1993); Haskil et al., Cell, 65, 1281-1289, 1991). The proteins of the IKB family mask the NF-KB nuclear translocation signal. The stimulation of the cell by various types of ligands, such as cytokines, the anti-CD40 ligand, lipopolysaccharide (LPS), oxidizing agents, mitogens, such as phorbol ester, viruses and many other stimulants, results in the activation of the IKB-kinase (IKK) complex, which in its turn will phosphorylate IKB at serine residues 32 and 34. Once phosphorylated, IKB will be subject to ubiquitinations resulting in its degradation by the proteasome (26S), thus making possible the release and the translocation of NF-KB into the nucleus, where it will become bonded to specific sequences in the promoters of target genes, thus resulting in their transcription.

In the IKB-kinase (IKK) complex, the main kinases are IKK1 (IKKα) and IKK2 (IKKβ), which are capable of directly phosphorylating the various classes of IKB. In this IKK complex, IKK2 is the dominant kinase (Mercurio et al., Mol Cell Biol, 19: 1526, 1999-, Zandi et al., Science, 28 1: 1 3) 60, 1998; Lee et al., Proc. Natl. Acad. Sci. USA, 95:93) 19, 1998). Among the genes regulated by NF-KB, many code for proinflammatory mediators, cytokines, cell adhesion molecules, acute phase proteins, which in their turn will also bring about activation of NF-KB by autocrine or paracrine mechanisms.

The inhibition of the activation of NF-KB appears to be very important in the treatment of inflammatory diseases.

In addition, NF-KB plays a role in the growth of normal cells but also of malignant cells.

The proteins produced by the expression of genes regulated by NF-KB comprise cytokines, chemokines, adhesion molecules, mediators of cell growth, of angiogenesis. Furthermore, various studies have shown that NF-KB plays an essential role in neoplastic transformations. For example, NF-KB can be associated with the transformation of cells in vitro and in vivo following overexpression, amplification, rearrangement or translocation events (Mercurio, R. and Manning, A. M. (1999), Oncogene, 18: 6163-6171). In some human lymphoid tumor cells, the genes coding for the various NF-KB members are rearranged or amplified. It has been shown that NF-KB can promote cell growth by bringing about the transcription of cyclin D, which, associated with the hyperphosphorylation of Rb, results in G1 to S phase transition and inhibition of apoptosis.

It has been shown that, in a large number of tumor cell lines, a constitutive NF-KB activity is found following the activation of IKK2. NF-KB is constitutively activated in Hodgkin's diseases and inhibition of NF-KB blocks the growth of these lymphomas. Moreover, inhibition of NF-KB by the expression of the repressor IKBa results in apoptosis of the cells expressing the oncogenic allele of H-Ras (Baldwin, J. Clin. Invest., 107: 241 (2001), Bargou et al., J. Clin. Invest., 100: 2961 (1997), Mayo et al., Science, 178: 1812 (1997)).

The constitutive NF-KB activity appears to contribute to oncogenesis through the activation of several antiapoptotic genes, such as Al/Bfi-1, IEX-1, MAP, which thus results in the suppression of the cell death pathway. Through the activation of cyclin D, NF-KB can promote the growth of tumor cells. The regulation of adhesion molecules and surface proteases suggests a role of NF-KB signaling in metastases.

NF-KB is involved in the induction of chemoresistance. NF-KB is activated in response to a certain number of chemotherapy treatments. It has been shown that the inhibition of NF-KB by the use of the superrepressor form of IKBa in parallel with the chemotherapy treatment increases the effectiveness of the chemotherapy in xenograft models.

The present invention thus especially relates to the products of formula (I):

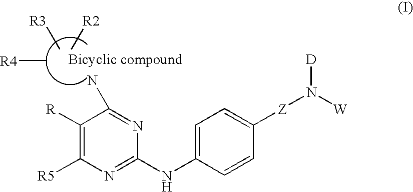

in which:
bicyclic compound represents an unsaturated or partially unsaturated bicyclic radical composed of 9 or 10 ring members, containing one or two nitrogen atoms, bearing the radicals R2, R3 and R4 and, optionally, additionally bearing an oxo functional group;
R represents a hydrogen atom or a halogen atom;
R2, R3 and R4, being identical or different, are chosen from a hydrogen atom, halogen atoms, the CN, CONH₂, CONHalk or CON(alk)₂ radical and alkyl and alkoxy radicals that are themselves optionally substituted by one or more halogen atoms or a CN, CONH₂, CONHalk, CON(alk)₂, OH or OCH₃ radical, it being understood that one or two of R2, R3 and R4 represent a hydrogen atom or else R2, R3 and R4 all represent methoxy;
R5 represents a hydrogen atom or a halogen atom;
Z represents CO or SO₂;
and the radical —N(D)(W) is such that:
a) either W represents -ring(Y) radical
and D represents a hydrogen atom, a cycloalkyl radical or an alkyl, alkenyl or alkynyl radical, all optionally substituted by one or more identical or different radicals chosen from halogen atoms, OR8 and NR8R9, the alkyl radicals represented by D in addition optionally being substituted by a saturated or unsaturated 5-membered heterocyclic radical attached via a carbon atom and optionally substituted by one or more radicals chosen from one or more halogen atoms and alkyl or alkoxy radicals;
and the ring (Y) is monocyclic or bicyclic, having from 4 to 10 ring members and being saturated or partially saturated with Y representing an oxygen atom O, a sulphur atom S, optionally oxidized by one or two oxygen atoms, or a radical chosen from NR10, C═O or its dioxolane as protective group for the carbonyl functional group, CF₂, CH—OR8 or CH—NR8R9;
it being understood that the ring (Y), when Y represents R10, can include a carbon bridge composed of 1 to 3 carbons,
R10 represents the hydrogen atom, a cycloalkyl radical or an alkyl, CH₂-alkenyl or CH₂-alkynyl radical, all optionally substituted by a naphthyl radical or by one or more identical or different radicals chosen from halogen atoms and hydroxyl, alkoxy, aryl and heteroaryl radicals, the alkyl radicals represented by R10 in addition being optionally substituted by a hydroxyl, NR8R9, CONR8R9, phosphonate, alkylthio, optionally oxidized to give sulphone, or heterocycloalkyl, all the aryl, heteroaryl and heterocycloalkyl radicals being optionally substituted;
b) or W and D form, with the nitrogen atom to which they are bonded, a ring (N)

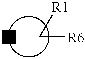

substituted on the same carbon atom by R1 and R6, containing 4 to 7 ring members, being saturated and possibly additionally incorporating a carbon bridge composed of 1 to 3 carbons,
being understood that R1 and R6 represent one of the 6 following alternatives i) to vi):
i) R1 represents -X1-R7 where X1 represents —(CH₂)ₘ— and R7 represents a heterocycloalkyl, aryl or heteroaryl ring, all optionally substituted;
and R6 represents a hydrogen atom, or hydroxyl, methyl, methoxy, —(CH₂)ₘOH, —CO—NRaRb, —CH₂—NraRb, —CO₂H and —CO₂alk radicals;
ii) R1 represents -X2-R7 where X2 represents:
—O—; —O—(CH₂)ₘ—; —CH(OH)—(CH₂)ₙ—; —CO—; —CO—NRc-;
—CO—NRc-O—; —CH(NRaRb)—; —C═NOH—; —C═N—NH₂—; —(CH2)ₙ₁-NRc-(CH₂)ₙ₂—; and R7 represents a heterocycloalkyl, aryl or heteroaryl ring, all optionally substituted;
and R6 represents hydrogen or a methyl radical;
iii) R1 represents —NRc-W where W represents a hydrogen atom or an alkyl radical containing 1 to 4 carbon atoms that is linear or branched starting from 3 carbon atoms optionally substituted by a radical chosen from —PO(OEt)₂, —OH, —Oalk, —CF₃, —CO—NR8R9 and SO₂-alk; and R6 represents hydrogen;
being understood that when W represents a hydrogen atom then z represents CO;
iv) R1 represents —CH₂—NRc-W where W represents a hydrogen atom or an alkyl radical containing 1 to 4 carbon atoms that is linear or branched starting from 3 carbon atoms and optionally substituted by a radical chosen from —PO(OEt)₂, —OH, —OEt, —CF₃, —CO—N(alk)₂ and SO₂-alk; and R6 represents hydrogen;
v) R1 represents —CO—N(Rc)-OR′c and R6 represents hydrogen;
vi) R1 represents X3-R7 where X3 represents —CH(OH)—(CH₂)n-; —CO—; —CH(NRaRb)—; —C═NOH—; —C═N—NH₂—;
and R7 represents a heterocycloalkyl, aryl or heteroaryl ring, all optionally substituted;
and R6 represents a hydrogen atom or hydroxyl, methyl, methoxy, —(CH₂)ₘOH, —CO—NRaRb, —CH₂—NraRb and —CO₂alk radicals;
where n, n1 and n2, being identical or different, represent an integer from 0 to 3;
m represents an integer from 1 to 3;
Rc and R′c, being identical or different, represent a hydrogen atom or an alkyl radical containing from 1 to 4 carbon atoms optionally substituted by one or more halogen atoms;
R8 represents the hydrogen atom or alkyl, cycloalkyl or heterocycloalkyl radicals, themselves optionally substituted by one or more radicals chosen from halogen atoms and hydroxyl, alkoxy, NH₂, NHalkyl, N(alkyl)₂, —CONH₂, —CONHalkyl or —CON(alkyl)₂ radicals, the alkyl radicals represented by R8 in addition being optionally substituted by a phosphonate radical, an alkylthio radical, optionally oxidized to sulphone, or by a saturated or unsaturated, optionally substituted, aryl or heterocyclic radical;
NR8R9 is such that either R8 and R9, which are identical or different, are chosen from the values of R8 or R8 and R9 form, with the nitrogen atom to which they are bonded, a cyclic amine which can optionally include one or two other heteroatoms chosen from O, S, N or NRc, the cyclic amine thus formed being itself optionally substituted;
all the above aryl, naphthyl, phenyl, heterocyclic, heterocycloalkyl and heteroaryl radicals and also the cyclic amine which can be formed by R8 and R9 with the nitrogen atom to which they are bonded being themselves optionally substituted by one or more identical or different radicals chosen from halogen atoms; hydroxyl; cyano; NR8R9 radicals; and alkyl, cycloalkyl, alkoxy, phenyl, heterocycloalkyl and heteroaryl radicals, themselves optionally substituted by one or more identical or different radicals chosen from halogen atoms and hydroxyl, alkoxy, alkyl, hydroxyalkyl, alkoxyalkyl, CN, CF₃, OCF₃, or NRaRb radicals;
NRaRb is such that either Ra and Rb, being identical or different, represent a hydrogen atom or an alkyl radical containing from 1 to 4 carbon atoms or a cycloalkyl radical, these alkyl and cycloalkyl radicals being optionally substituted by one or more identical or different radicals chosen from halogen atoms and hydroxyl, alkoxy, NH₂, NHalkyl and N(alkyl)₂ radicals; or Ra and Rb form, with the nitrogen atom to which they are bonded, a cyclic amine which may optionally contain one or two other heteroatoms chosen from O, S, N or NRc, the cyclic amine thus formed being itself optionally substituted by one or more identical or different radicals chosen from halogen atoms and oxo; hydroxyl; or alkyl radicals themselves optionally substituted by one or more halogen atoms; or else by a methyl radical and a hydroxyl radical on the same carbon;
all the heterocyclic, heterocycloalkyl and heteroaryl radicals above being composed of 4 to 10 ring members (except where specified) and containing 1 to 4 heteroatoms chosen, where appropriate, from O, optionally oxidized S, N and NRc;
the said products of the formula (I) being in all the possible isomeric forms, racemic, enantiomeric and diastereoisomeric, and also the addition salts with inorganic and organic acids of the said products of formula (I).

The present invention thus especially relates to the products of formula (I) as defined above or below in which R2, R3, R4, R5, Z and the —N(D)(W) radical have the meanings indicated above or below and R represents a halogen atom;

the said products of formula (I) being in all the possible isomeric forms, racemic, enantiomeric and diastereoisomeric, and also the addition salts with inorganic and organic acids of the said products of formula (I).

The present invention thus especially relates to the products of formula (I) as defined above or below in which R2, R3, R4, R5 and Z and the —N(D)(W) radical have the meanings indicated in any one of the other claims and R represents a hydrogen atom;

the said products of formula (I) being in all the possible isomeric forms, racemic, enantiomeric and diastereoisomeric, and also the addition salts with inorganic and organic acids of the said products of formula (I).

The present invention thus especially relates to the products of formula (I) as defined above or below in which R, R5 and Z and the —N(D)(W) radical have the meanings indicated in any one of the other claims, and R2, R3 and R4, which may be identical or different, are chosen from a hydrogen atom, halogen atoms, the CN radical and the alkyl and alkoxy radicals themselves optionally substituted by one or more halogen atoms or a CN, CONH2, CONHalk or CON(alk)2 radical, it being understood that one or two of R2, R3 and R4 represent hydrogen or else R2, R3 and R4 all represent methoxy;

the said products of formula (I) being in all the possible isomeric forms, racemic, enantiomeric and diastereoisomeric, and also the addition salts with inorganic and organic acids of the said products of formula (I).

The present invention especially relates to products of formula (I) such as defined above or below in which R, R5, Z and the —N(D)(W) radical have the meanings indicated above or below and R2, R3, R4 are such that one of R2, R3 and R4 represents a CN or CH₂—CN radical and the other two of R2, R3 and R4 are chosen from the other values defined for these radicals, i.e. from a hydrogen atom, halogen atoms, the CN, CONH₂, CONHalk, CON(alk)₂ radical, and the alkyl and alkoxy radicals themselves optionally substituted by one or more halogen atoms or a CN, CONH₂, CONHalk, CON(alk)₂, OH or OCH₃ radical, being understood that one or two of R2, R3 and R4 represent a hydrogen atom, the said products of formula (I) being in all the possible isomeric forms, racemic, enantiomeric and diastereoisomeric, and also the addition salts with inorganic and organic acids of the said products of formula (I). It is obvious that in the latter case, R2, R3 and R4 cannot all represent methoxy.

One subject of the present invention is the products of formula (I) as defined above and below in which:

Bicyclic compound represents a bicyclic radical consisting of nine or ten ring members, unsaturated or partially unsaturated, containing one or two nitrogen atoms, bearing the radicals R2, R3 and R4 and in addition bearing, optionally, an oxo functional group,
R has the meaning indicated above or below,
R2, R3 and R4, which are identical or different, represent a hydrogen atom, a halogen atom, CN or an alkyl or alkoxy radical optionally substituted by one or more halogen atoms;
R5 represents a hydrogen atom or a halogen atom;
Z represents CO or SO₂;
and the —N(D)(W) radical is such that:
a) either W represents -ring(Y) radical
and D represents a hydrogen atom, a cycloalkyl radical or an alkyl, alkenyl or alkynyl radical, all optionally substituted by one or more identical or different radicals chosen from halogen atoms, OR8 and NR8R9, the alkyl radicals represented by D in addition optionally being substituted by a saturated or unsaturated 5-membered heterocyclic radical attached via a carbon atom or a nitrogen atom and optionally substituted by one or more radicals chosen from halogen atoms and alkyl or alkoxy radicals;
and the ring (Y) is monocyclic or bicyclic, having from 4 to 10 ring members and being saturated or partially saturated with Y representing an oxygen atom O, a sulphur atom S, optionally oxidized by one or two oxygen atoms, or a radical chosen from NR10, C═O or its dioxolane as protective group for the carbonyl functional group, CF₂, CH—OR8 or CH—NR8R9;
it being understood that the ring Y, when Y represents R10, can include a carbon bridge composed of 1 to 3 carbons,
R10 represents the hydrogen atom, a cycloalkyl radical or an alkyl, CH₂-alkenyl or CH₂-alkynyl radical, all optionally substituted by a naphthyl radical or by one or more identical or different radicals chosen from halogen atoms and hydroxyl, alkoxy, aryl and heteroaryl radicals, the alkyl radicals represented by R10 in addition being optionally substituted by a hydroxyl, NR8R9, CONR8R9, phosphonate, alkylthio, optionally oxidized to give sulphone or heterocycloalkyl, radical, all the aryl, heteroaryl and heterocycloalkyl radicals being optionally substituted;
b) or W and D form, with the nitrogen atom to which they are bonded, a ring (N)

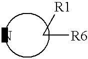

substituted on the same carbon atom by R1 and R6, containing 4 to 7 ring members, being saturated and possibly additionally incorporating a carbon bridge composed of 1 to 3 carbons,
it being understood that R1 and R6 represent one of the 5 following alternatives i) to v):
i) R1 represents -X1-R7 where X1 represents —(CH₂)ₘ— and R7 represents a heterocycloalkyl, aryl or heteroaryl ring, all optionally substituted;
and R6 represents a hydrogen atom, or hydroxyl, —(CH₂)ₘOH, —CO—NRaRb, —CH₂—NRaRb, —CO₂H and —CO₂alk radicals;
ii) R1 represents -X2-R7 where X2 represents:
—O—; —O—(CH₂)ₘ—; —CH(OH)—(CH₂)ₙ—; —CO—; —CO—NRc-;

### —CO—NRc-O—; —CH(NRaRb)—; —C═NOH—; —C═N—NH₂—;

—(CH2)ₙ₁-NRc-(CH₂)ₙ₂—; and R7 represents a heterocycloalkyl, aryl or heteroaryl ring, all optionally substituted;
and R6 represents hydrogen;
iii) R1 represents —NRc-A where A represents a hydrogen atom or an alkyl radical containing 1 to 4 carbon atoms that is linear or branched starting from 3 carbon atoms optionally substituted by a radical chosen from —PO(OEt)₂, —OH, —Oalk, —CF₃, —CO—NR8R9 and SO₂-alk; and R6 represents hydrogen;
being understood that when A represents a hydrogen atom then z represents CO;
iv) R1 represents —CH₂—NRc-A where A represents a hydrogen atom or an alkyl radical containing 1 to 4 carbon atoms that is linear or branched starting from 3 carbon atoms and optionally substituted by a radical chosen from —PO(OEt)₂, —OH, —OEt, —CF₃, —CO—N(alk)₂ and SO₂-alk; and R6 represents hydrogen;
v) R1 represents —CO—N(Rc)-OR′c and R6 represents hydrogen;
where n, n1 and n2, being identical or different, represent an integer from 0 to 3;
m represents an integer from 1 to 3;
Rc and R′c, being identical or different, represent a hydrogen atom or an alkyl radical containing from 1 to 4 carbon atoms optionally substituted by one or more halogen atoms;
R8 represents the hydrogen atom or alkyl, cycloalkyl or heterocycloalkyl radicals, themselves optionally substituted by one or more radicals chosen from halogen atoms and hydroxyl, alkoxy, NH₂, NHalkyl, N(alkyl)₂, —CONH₂, —CONHalkyl or —CON(alkyl)₂ radicals, the alkyl radicals represented by R8 in addition being optionally substituted by a phosphonate radical, an alkylthio radical, optionally oxidized to sulphone, or by a saturated or unsaturated, optionally substituted, heterocyclic or aryl radical;
NR8R9 is such that either R8 and R9, which are identical or different, are chosen from the values of R8 or R8 and R9 form, with the nitrogen atom to which they are bonded, a cyclic amine which can optionally include one or two other heteroatoms chosen from O, S, N or NRc, the cyclic amine thus formed being itself optionally substituted;
all the above aryl, naphthyl, phenyl, heterocyclic, heterocycloalkyl and heteroaryl radicals and also the cyclic amine which can be formed by R8 and R9 with the nitrogen atom to which they are bonded being themselves optionally substituted by one or more identical or different radicals chosen from halogen atoms; hydroxyl; cyano; NR8R9 radicals; and alkyl, cycloalkyl, alkoxy, phenyl, heterocycloalkyl and heteroaryl radicals, themselves optionally substituted by one or more identical or different radicals chosen from halogen atoms and hydroxyl, alkoxy, alkyl, hydroxyalkyl, alkoxyalkyl, CN, CF₃, OCF₃, or NRaRb radicals;
NRaRb is such that either Ra and Rb, being identical or different, represent a hydrogen atom or an alkyl radical containing from 1 to 4 carbon atoms or a cycloalkyl radical, these alkyl and cycloalkyl radicals being optionally substituted by one or more identical or different radicals chosen from halogen atoms and hydroxyl, alkoxy, NH₂, NHalkyl and N(alkyl)₂ radicals; or Ra and Rb form, with the nitrogen atom to which they are bonded, a cyclic amine which may optionally contain one or more other heteroatoms chosen from O, S, N or NRc, the cyclic amine thus formed being itself optionally substituted by one or more identical or different radicals chosen from halogen atoms and alkyl radicals themselves optionally substituted by one or more halogen atoms;
all the heterocyclic, heterocycloalkyl and heteroaryl radicals above being composed of 4 to 10 ring members (except where specified) and containing 1 to 4 heteroatoms chosen, where appropriate, from O, S optionally oxidized, N and NRc;
the said products of formula (I) being in all the possible isomeric forms, racemic, enantiomeric and diastereoisomeric, and also the addition salts with inorganic and organic acids of the said products of formula (I).

Thus, one subject of the present invention is the products of formula (I) such as defined above corresponding to the formula (IA):

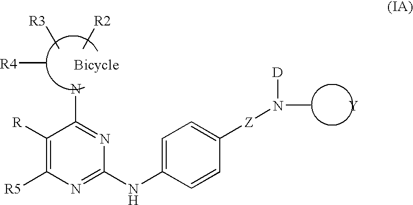

in which bicyclic compound, R, R2, R3, R4, R5, z, D and ring (Y) have the meanings indicated above or below,
the said products of formula (I) being in all the possible isomeric forms, racemic, enantiomeric and diastereoisomeric, and also the addition salts with inorganic and organic acids of the said products of formula (I).

The present invention thus especially relates to the products of formula (I) such as defined above corresponding to the formula (IA) in which R, R2, R3, R4, R5, Z and D are chosen from the meanings indicated above or below and ring(Y) may be chosen from any one of the following values:
When ring(Y) is such that Y represents C—OH, CF₂, CH—OR8 or CH—NR8R9, the ring formed can in particular be a cyclobutyl, cyclopentyl, cyclohexyl or cycloheptyl and particularly a cyclohexyl, these radicals thus being substituted, in particular in the para position, respectively by OH, 2F, the OR8 radical or the NR8R9 radical in which R8 and R9 are chosen from the meanings defined above.
When ring(Y) is such that Y represents NR10, the ring formed can in particular be an azetidinyl, pyrrolidinyl or piperidinyl radical with the nitrogen atom N in the para or in the meta position, which thus carries the substituent R10 as defined above: thus ring(Y) may represent a pyrrolidinyl or piperidinyl radical optionally substituted on the nitrogen atom by R10 which may represent an alkyl radical optionally substituted by a hydroxyl, —NR8R9, —CO—NR8R9, phosphonate or alkylthio, optionally oxidized to sulphone, radical.
When ring(Y) such that Y represents NR10 includes a carbon bridge composed of 1 to 3 carbons, the ring formed can in particular be the 8-azabicyclo[3.2.1]octan-3-yl ring or also a ring chosen from the following rings: N,9-dimethyl-9-azabicyclo[3.3.1]nonan-3-yl, N,6-dimethyl-6-azabicyclo[3.2.1]octan-3-yl, N,3-dimethyl-3-azabicyclo[3.2.1]octan-8-yl or also N,3-dimethyl-3-azabicyclo[3.3.1]nonan-9-yl.
When ring(Y) is such that Y represents NR10, the ring formed can in particular be a bicyclic radical, such as, for example, quinolizinyl or indolizinyl;
When ring(Y) is such that Y represents S, the ring formed can in particular be a tetrahydrothiopyranyl or a tetrahydrothiophene: when ring(Y) is such that Y represents SO₂, the ring formed can in particular be a dioxidotetrahydro-3-thiophene.
When ring(Y) is such that Y represents O, the ring formed can in particular be a tetrahydrofuran or tetrahydropyran. When ring(Y) is such that Y represents the dioxolane of C═O, the ring formed can in particular be dioxaspiro[4.5]dec-8-yl.

Mention may similarly be made of:
ring(Y) such that Y represents —NR10 where R10 represents H;
ring(Y) such that Y represents —NR10 where R10 represents CH₃;
ring(Y) such that Y represents —NR10 where R10 represents cycloalkyl such as, in particular, cyclopropyl;
ring(Y) such that Y represents —NR10 where R10 represents an alkyl radical, especially CH₃, C₂H₅ or C₃H₇ substituted by a phosphonate;
ring(Y) such that Y represents —NR10 where R10 represents an alkyl radical, especially CH₃, C₂H₅ or C₃H₇ substituted by an alkylthio such as S—CH₃ or S—C₂H₅ with S optionally oxidized to sulphone to form, for example, SO₂—CH₃ or SO₂—C₂H₅;
ring(Y) such that Y represents —NR10 where R10 represents alkyl such as, in particular, CH₃ or C₂H₅ substituted by one or more radicals chosen from halogen atoms such as, in particular, F, and phenyl and monocyclic or bicyclic heterocyclic radicals, the phenyl and heterocyclic radicals themselves optionally being substituted by one or more radicals chosen from halogen atoms and alkyl, alkoxy, OH, CN, CF₃, NH₂, NHalk and N(alk)₂ radicals: from among these heterocycles which may bear R10, mention may especially be made of unsaturated heterocycles having 5 ring members that contain one to four heteroatoms chosen from N, O and S: thus R10 may represent, in particular, the —CH₂-thienyl, —CH₂-thiazolyl (N,S), —CH₂-thiadiazolyl (N,N,S), —CH₂-furanyl (O), —CH₂-pyrazolyl (N,N), —CH₂-isoxazolyl (N,O), —CH₂-pyrrolyl (NH, NCH₃) radicals, these radicals, especially pyrazolyl, isoxazolyl, pyrrolyl or tetrazolyl, being themselves optionally substituted, especially by an alkyl containing 1 to 3 carbon atoms such as, in particular, CH₃ or C₂H₅.

R10 may also bear heterocycles such as defined above such as the pyridinyl (with N of the pyridine at 3 different positions); 2,3-dihydro-1H-indolyl; quinolyl; isoquinolyl; pyrimidinyl; 2,3-dihydrobenzofuranyl; [1,8]naphthyridinyl; pyridinyl-N-oxide; 4-benzo[1,2,5]oxadiazolyl; 2,3-dihydrobenzofuranyl radicals.
Ring(Y) such that Y represents CH—NR8R9 with NR8R9 such that R8 represents a hydrogen atom or an alkyl radical such as, in particular, CH₃ and R9 represents a linear or branched alkyl radical such as, in particular, CH₃, C₂H₅ or —CH₂— or —CH(CH₃)— or —CH(CH₃)—CH₂— substituted either by an optionally substituted, saturated or unsaturated, monocyclic or bicyclic heterocycle or by an optionally substituted phenyl radical. Among the heterocycles which bear R9, mention may especially be made of the following radicals: pyridine (with N of the pyridine at 3 different positions); 2,3-dihydro-1H-indolyl; quinolyl; isoquinolyl; pyrimidinyl; 2,3-dihydrobenzofuranyl; [1,8]naphthyridine; 4-benzo[2,1,3]oxadiazolyl; benzo-[2,1,3]thiadiazolyl;
   Such heterocycles are optionally substituted by one or more radicals such as defined above or below.

The present invention thus especially relates to products of formula (I) such as defined above corresponding to the formula (IA) in which R, R2, R3, R4, R5 and Z and ring(Y) are chosen from the meanings indicated above or below and D may be chosen from any one of the following values:
D represents a hydrogen atom or a linear or branched alkyl radical containing 1 to 6 carbon atoms optionally substituted by an NH₂, NHalk, N(alk)₂ radical or by a saturated or unsaturated heterocycle, preferably a monocyclic compound having 5 or 6 ring members such as defined above and optionally substituted as indicated above or below;
D represents a hydrogen atom or a linear or branched alkyl radical containing from 1 to 5 carbon atoms optionally substituted by NH₂ or else D represents this alkyl radical substituted by a saturated or unsaturated heterocycle, preferably a monocyclic compound having 5 ring members itself optionally substituted as indicated above or below;
D is chosen from the values defined above and ring(Y) represents a cyclohexyl radical substituted by an NR8R9 radical as defined above;
D represents a CH₃ radical optionally substituted by a saturated or unsaturated heterocycle as defined above and R10 represents a CH₃ radical;
D represents a hydrogen atom or a CH₃ radical and ring(Y) represents a piperidine or an 8-azabicyclo[3.2.1]octan-3-yl ring substituted on their nitrogen atom by R10 with R10 as defined above.

More precisely:
D represents H;
D represents CH₃;
D represents alkenyl (3C) radicals, such as allyl, or alkynyl (3C) radicals, such as propargyl;
D represents alkyl and in particular CH₃, C₂H₅, C₃H₇, substituted by one or more identical or different radicals chosen from halogen atoms and NH₂, NH(alk), N(alk)₂, NH—CH₂—CH₂OH, NH—CH₂—C₃H₇—OH, NH(CH₂—CF₃), alkoxy or OH radicals or a saturated heterocycle, such as, for example, pyrrolidinyl, piperidinyl, morpholinyl or tetrahydrofuranyl, or an unsaturated heterocycle, such as, in particular, those defined above for R10.

Thus, one subject of the present invention is the products of such as defined above or below corresponding to the formula (IA) in which bicyclic compound, R, R2, R3, R4, R5 and z have the meanings indicated above or below, D represents a hydrogen atom or a linear or branched alkyl radical containing from 1 to 4 carbon atoms optionally substituted by NH₂ and especially CH₃ and ring(Y) is such that Y represents NR10 where R10 represents a linear or branched alkyl radical containing 1 to 6 carbon atoms optionally substituted by a radical chosen from halogen atoms and hydroxyl, phosphonate, sulphone, phenyl and saturated or unsaturated heterocyclic, monocyclic or bicyclic radicals, these phenyl and heterocyclic radicals being themselves optionally substituted as indicated above or below,

the said products of formula (I) being in all the possible isomeric forms, racemic, enantiomeric and diastereoisomeric, and also the addition salts with inorganic and organic acids of the said products of formula (I).

Thus, one subject of the present invention is the products of formula (I) such as defined above or below corresponding to the formula (IA) in which bicyclic compound, R, R2, R3, R4, R5 and z have the meanings indicated above or below,

D represents a linear or branched alkyl radical containing from to 4 carbon atoms optionally substituted by NH₂ and especially CH₃ and ring(Y) is such that Y represents NR8R9 in which R8 represents a hydrogen atom or an alkyl radical and R9 represents a linear or branched alkyl radical containing 1 to 6 carbon atoms optionally substituted by a radical chosen from halogen atoms and hydroxyl, phosphonate, sulphone, phenyl and saturated or unsaturated heterocyclic, monocyclic or bicyclic radicals, these phenyl and heterocyclic radicals being themselves optionally substituted as indicated above or below, the said products of formula (I) being in all the possible isomeric forms, racemic, enantiomeric and diastereoisomeric, and also the addition salts with inorganic and organic acids of the said products of formula (I).

Thus, one subject of the present invention is the products of formula (I) such as defined above or below corresponding to formula (IB):

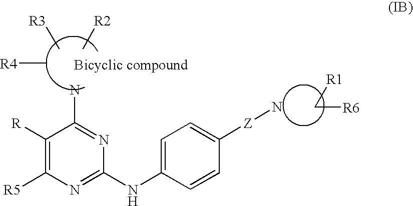

in which bicyclic compound, R, R1, R2, R3, R4, R5, R6, Z and ring (N) have the meanings indicated above or below, the said products of formula (I) being in all the possible isomeric forms, racemic, enantiomeric and diastereoisomeric, and also the addition salts with inorganic and organic acids of the said products of formula (I).

Thus, one subject of the present invention is the products of the formula (I) as defined above or below in which:

bicyclic compound represents an unsaturated or partially unsaturated bicyclic radical composed of 9 or 10 ring members, containing one or two nitrogen atoms, bearing the radicals R2, R3 and R4 and, optionally, additionally bearing an oxo functional group;
R has the meaning indicated above or below,
R2, R3 and R4, which are identical or different, represent a hydrogen atom, a halogen atom, CN or an alkyl or alkoxy radical optionally substituted by one or more halogen atoms;
R5 represents a hydrogen atom or a halogen atom;
Z represents CO or SO₂;
the ring (N) i.e.

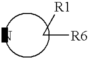

being substituted on the same carbon atom by R1 and R6, containing 4 to 7 ring members, being saturated and possibly additionally incorporating a carbon bridge composed of 1 to 3 carbons, where R1 and R6 such as defined above or below, the said products of formula (I) being in all the possible isomeric forms, racemic, enantiomeric and diastereoisomeric, and also the addition salts with inorganic and organic acids of the said products of formula (I).

Thus, one subject of the present invention is the products of formula (I) such as defined above or below corresponding to the formula (IB) in which R2, R3, R4, R5, Z and the ring(N) have the meanings indicated above or below and R1 and R6 are such that R1 represents -X1-R7 where X1 represents —(CH₂)ₘ— and R7 represents a heterocycloalkyl, aryl or heteroaryl ring, all optionally substituted;

and R6 represents a hydrogen atom or hydroxyl, —(CH₂)ₘOH, —CO—NRaRb, —CH₂—NRaRb—CO2H, and —CO₂alk radicals;
with m, n and NRaRb such as defined above or below and the heterocycloalkyl, aryl and heteroaryl radicals being optionally substituted by one or more identical or different radicals, such as defined above or below,
the said products of formula (I) being in all the possible isomeric forms, racemic, enantiomeric and diastereoisomeric, and also the addition salts with inorganic and organic acids of the said products of formula (I).

Thus, one subject of the present invention is the products of formula (I) such as defined above or below corresponding to the formula (IB) in which bicyclic compound, R, R2, R3, R4, R5, Z and the ring(N) have the meanings indicated above or below and R1 and R6 are such that R1 represents -X2-R7 where X2 represents:

—O—; —O—(CH₂)ₘ—; —CH(OH)—(CH₂)ₙ—; —CO—; —CO—NRc-;
—CO—NRc-O—; —CH(NRaRb)—; —C═NOH—; —C═N—NH₂—; —(CH2)ₙ₁-NRc-(CH₂)ₙ₂—; and R7 represents a heterocycloalkyl, aryl or heteroaryl ring, all optionally substituted;
and R6 represents hydrogen;
with n, n1, n2, Rc and NRaRb such as defined above or below and the heterocycloalkyl, aryl and heteroaryl radicals being optionally substituted by one or more identical or different radicals such as defined above or below,
the said products of formula (I) being in all the possible isomeric forms, racemic, enantiomeric and diastereoisomeric, and also the addition salts with inorganic and organic acids of the said products of formula (I).

Thus, one subject of the present invention is the products of formula (I) corresponding to the formula (IB) such as defined above or below in which bicyclic compound, R, R2, R3, R4, R5, Z and the ring(N) have the meanings indicated above or below and R1 and R6 are such that:

either R1 represents —NRc-A where A represents a hydrogen atom or an alkyl radical containing 1 to 4 carbon atoms that is linear or branched starting from 3 carbon atoms optionally substituted by a radical chosen from —PO(OEt)₂, —OH, —Oalk, —CF₃, —CO—NR8R9 and SO₂-alk; and R6 represents hydrogen;
or R1 represents CH₂—NRc-A where A represents a hydrogen atom or an alkyl radical containing 1 to 4 carbon atoms that is linear or branched starting from 3 carbon atoms and optionally substituted by a radical chosen from —PO(OEt)₂, —OH, —OEt, —CF₃, —CO—N(alk)₂ and SO₂-alk;
and R6 represents hydrogen;
or R1 represents —CO—N(Rc)-OR′c and R6 represents hydrogen;
with Rc, R′c and NR8R9 such as defined above or below, the said products of formula (I) being in all the possible isomeric forms, racemic, enantiomeric and diastereoisomeric, and also the addition salts with inorganic and organic acids of the said products of formula (I).

When the ring(N) of the products of formula (I) corresponding to the formula (IB) contains a carbon bridge composed of 1 to 3 carbons, the ring formed may especially be the 8-azabicyclo[3.2.1]oct-3-yl ring, or else a ring chosen from the following: 9-azabicyclo[3.3.1]nonan-3-yl, 6-azabicyclo[3.2.1]octan-3-yl, 3-azabicyclo[3.2.1]octan-8yl or else 3-azabicyclo[3.3.1]nonan-9-yl.

In the products of formula (I) and in that which follows, the terms indicated have the meanings which follow:
the term “halogen” denotes the fluorine, chlorine, bromine or iodine atoms and preferably the fluorine, chlorine or bromine atoms;
the term “alkyl radical” denotes a linear or branched radical including at most 6 carbon atoms and in particular the methyl, ethyl, propyl, isopropyl, n-butyl, isobutyl, sec-butyl, tert-butyl, pentyl, isopentyl, sec-pentyl, tert-pentyl, neopentyl, hexyl, isohexyl, sec-hexyl or tert-hexyl radicals and also their linear or branched positional isomers;
the term “hydroxyalkyl radical” denotes the alkyl radicals indicated above substituted by one or more hydroxyl radicals;
the term “alkenyl radical” denotes a linear or branched radical including at most 6 carbon atoms and preferably 4 carbon atoms chosen, for example, from the following values: ethenyl or vinyl, propenyl or allyl, 1-propenyl, n-butenyl, isobutenyl, 3-methylbut-2-enyl, n-pentenyl or hexenyl, and also their linear or branched positional isomers: mention is more particularly made, among the alkenyl values, of the allyl or butenyl values;
the term “alkynyl radical” denotes a linear or branched radical including at most 6 carbon atoms and preferably 4 carbon atoms chosen, for example, from the following values: ethynyl, propynyl or propargyl, butynyl, n-butynyl, isobutynyl, 3-methylbut-2-ynyl, pentynyl or hexynyl, and also their linear or branched positional isomers: mention is more particularly made, among the alkynyl values, of the propargyl value;
the term “alkylene radical” denotes a linear or branched divalent radical including at most 6 carbon atoms resulting from the above alkyl radical and thus chosen, for example, from the methylene, ethylene, propylene, isopropylene, butylene, isobutylene, sec-butylene or pentylene radicals;
the term “alkoxy radical” denotes a linear or branched radical including at most 6 carbon atoms chosen, for example, from among methoxy, ethoxy, propoxy, isopropoxy, butoxy, linear, secondary or tertiary, pentoxy, hexoxy and heptoxy, and also their linear or branched positional isomers;
the term “cycloalkyl radical” denotes a monocyclic or bicyclic carbocyclic radical including from 3 to 7 ring members and denotes in particular the cyclopropyl, cyclobutyl, cyclopentyl, cyclohexyl and cycloheptyl radicals;
the term “aryl radical” denotes unsaturated carbocyclic radicals which are monocyclic or composed of fused rings. Mention may in particular be made, as examples of such aryl radicals, of the phenyl or naphthyl radicals;
the term “heterocyclic radical” denotes a saturated carbocyclic (heterocycloalkyl) radical or a partially or completely unsaturated (heteroaryl) carbocyclic radical composed of 4 to 10 ring members interrupted by one or three identical or different heteroatoms chosen from the oxygen, nitrogen or sulphur atoms:
   mention may in particular be made, among 5-membered heteroaryl radicals, of the radicals including one to four heteroatoms chosen from N, optionally oxidized, O and S, optionally oxidized, mention may be made of the radicals 2-thienyl, 3-thienyl, dioxidothienyl, -thiazolyl (N,S), -furyl (O), 2-furyl, pyrrolyl (NH, NCH₃), isothiazolyl, diazolyl, thiadiazolyl (N,N,S), 1,3,4-thiadiazolyl, oxazolyl, oxadiazolyl, isoxazolyl (N,O), 3-isoxazolyl, 4-isoxazolyl, imidazolyl or pyrazolyl (N,N), triazolyl or tetrazolyl groups and more particularly the oxazolyl, isoxazolyl (N,O) or pyrazolyl radicals; all these rings optionally being substituted by one or more radicals as defined above or below, these substituents, of course, being located at the positions chemically acceptable for each of these rings;
   mention may in particular be made, among 6-membered heteroaryl radicals, of the pyridyl, such as 2-pyridyl, 3-pyridyl and 4-pyridyl, pyridyl N-oxide, pyrimidinyl, pyridazinyl and pyrazinyl radicals;
   mention may more particularly be made, among fused heterocyclyl radicals, of the benzothienyl, benzofuryl, benzofuranyl, benzoxazolyl, indazolyl, indolyl, indolinyl, 2-oxoindolinyl, quinolyl such as 4-quinolyl, 5-quinolyl, isoquinolyl, azaindolyl such as 4-azaindolyl, 3-azaindolyl, imidazo[4,5]pyridyl, indolizinyl benzimidazolyl, benzothiazolyl, naphthyridinyl such as [1,8]naphthyridinyl; imidazo[4,5]pyridyl; quinazolinyl; 2,3-Dihydro-1H-indolyl; 2,3-Dihydrobenzofuranyl; 4-[(Benzo[1,2,5]oxadiazolyl; (2,3-Dihydro-benzofuranyl; radicals;
   mention may be made, as heterocycloalkyl (saturated), for example, of the oxiranyl, oxetanyl, tetrahydrofuranyl, dioxolanyl, dithiolanyl, tetrahydropyranyl, dioxanyl, aziridinyl, azetidinyl, pyrrolidinyl, piperidinyl, azepinyl, diazepinyl, piperazinyl, morpholinyl, thiomorpholinyl, dioxidomorpholinyl or imidazolidinyl radicals; mention may more particularly be made of the pyrrolidinyl, piperidinyl, azepinyl, piperazinyl or morpholinyl radicals;
   all the cyclic radicals being optionally substituted as indicated above or below;
the terms “alkylamino or NH(alk) radical” and “dialkylamino or N(alk)₂ radical” thus denote amino NH₂ radicals respectively substituted by one or two linear or branched alkyl radicals, identical or different in the case of dialkylamino, chosen from the alkyl radicals as defined above and optionally substituted as indicated above or below: mention may be made, for example, of the methylamino, ethylamino, propylamino or butylamino radicals or the dimethylamino, diethylamino or methylethylamino radicals;
the term “cycloalkylamino radical” thus denotes an amino radical substituted in particular by a cycloalkyl radical chosen from the radicals defined above: mention may thus be made, for example, of the cyclopropylamino, cyclobutylamino, cyclopentylamino or also cyclohexylamino radicals;
the term “cyclic amine” denotes a monocyclic or bicyclic radical including from 3 to 10 ring members in which at least one carbon atom is replaced by a nitrogen atom, it being possible for this cyclic radical also to include one or more other heteroatoms chosen from O, S, SO₂, N or NRc with Rc as defined above: mention may be made, as examples of such cyclic amines, for example, of the pyrrolyl, piperidyl, morpholinyl, piperazinyl, pyrrolidinyl or azetidinyl radicals. Mention may more particularly be made of the piperidinyl, morpholinyl, piperazinyl, pyrrolidinyl or azetidinyl radicals optionally substituted as indicated above, especially by an oxo or hydroxyl or else hydroxyl and methyl radical on the same carbon.

The term “patient” denotes human beings but also other mammals. The term “prodrug” denotes a product which can be converted in vivo by metabolic mechanisms (such as hydrolysis) to a product of formula (I). For example, an ester of a product of formula (I) comprising a hydroxyl group can be converted by hydrolysis in vivo to its mother molecule.

Mention may be made, as examples of esters of products of formula (I) comprising a hydroxyl group, such as of the acetates, citrates, lactates, tartrates, malonates, oxalates, salicylates, propionates, succinates, fumarates, maleates, methylenebis(β-hydroxynaphthoates), gentisates, isethionates, di(p-toluoyl)tartrates, methanesulphonates, ethanesulphonates, benzenesulphonates, p-toluenesulphonates, cyclohexylsulfamates and quinates.

Esters of products of formula (I) which are particularly useful comprising a hydroxyl group can be prepared from acid residues, such as those described by Bundgaard et al., J. Med. Chem., 1989, 32, page 2503-2507: these esters include in particular (aminomethyl)benzoates which are substituted, dialkylaminomethylbenzoates in which the two alkyl groups can be bonded together or can be interrupted by an oxygen atom or by an optionally substituted nitrogen atom or an alkylated nitrogen atom or also (morpholinomethyl)benzoates, e.g. 3- or 4-(morpholinomethyl)benzoates, and (4-alkylpiperazin-1-yl)benzoates, e.g. 3- or 4-(4-alkylpiperazin-1-yl)benzoates. When the products of formula (I) comprise an amino radical which can be salified by an acid, it is clearly understood that these acid salts also form part of the invention.

The addition salts with inorganic or organic acids of the products of formula (I) can, for example, be the salts formed with hydrochloric, hydrobromic, hydriodic, nitric, sulphuric, phosphoric, propionic, acetic, trifluoroacetic, formic, benzoic, maleic, fumaric, succinic, tartaric, citric, oxalic, glyoxylic, aspartic or ascorbic acids, alkylmonosulphonic acids, such as, for example, methanesulphonic acid, ethanesulphonic acid or propanesulphonic acid, alkyldisulphonic acids, such as, for example, methanedisulphonic acid or α,β-ethanedisulphonic acid, arylmonosulphonic acids, such as benzosulphonic acid, and aryldisulphonic acids.

It may be remembered that stereoisomerism may be defined in its broad sense as the isomerism of compounds having the same expanded formulae but the various groups of which are positioned differently in space, such as, for example enantiomerism. However, there exists another type of stereoisomerism due to the different spatial arrangements of fixed substituents, either on double bonds or on rings, which are often referred to as E/Z geometrical isomerism or cis-trans isomerism or diastereoisomerism. The term “stereoisomer” is used in the present patent application in its broadest sense and thus relates to all the compounds indicated above.

The present invention has in particular as subject-matter the products of formula (I) such as defined above or below corresponding to the formula (IA) in which:

bicyclic compound represents an unsaturated or partially unsaturated bicyclic radical composed of 9 ring members, containing one or two nitrogen atoms, bearing the radicals R2, R3 and R4 and, optionally, additionally bearing an oxo functional group;
R has the meaning indicated above or below;
R2, R3 and R4, which are identical or different, represent a hydrogen atom, a halogen atom, CN or an alkyl or alkoxy radical that are themselves optionally substituted by one or more halogen atoms;
R5 represents a hydrogen atom or a halogen atom;
D represents a hydrogen atom, a cycloalkyl radical or an alkyl radical optionally substituted by one or more identical or different radicals chosen from the halogen atoms, OR8 and NR8R9;
the ring(Y), being monocyclic or bicyclic, having from 4 to 10 ring members and being saturated or partially saturated with Y representing an oxygen atom O, a sulphur atom S, optionally oxidized by one or two oxygen atoms, or a radical chosen from NR10, C═O, CF₂, CH—OR8 or CH—NR8R9;
R10 represents a hydrogen atom or an alkyl radical optionally substituted by one or more identical or different radicals chosen from halogen atoms and hydroxyl, alkoxy, phenyl and heteroaryl radicals, the phenyl and heteroaryl radicals being themselves optionally substituted by one or more identical or different radicals chosen from halogen atoms and hydroxyl, alkoxy, alkyl, hydroxyalkyl, alkoxyalkyl, CF₃, NH₂, NHalk or N(alk)₂ radicals;
The heteroaryl radicals being composed of 5 to 7 ring members and including 1 to 3 heteroatoms chosen from O, S, N and NRc;
R8 represents the hydrogen atom, linear or branched alkyl radicals including at most 4 carbon atoms or cycloalkyl radicals including from 3 to 6 ring members, the alkyl and cycloalkyl radicals being themselves optionally substituted by one or more identical or different radicals chosen from halogen atoms and hydroxyl, NH2, NHalk and N(alk)₂ radicals;
NR8R9 is such that either R8 and R9, which are identical or different, are chosen from the values of R8 or R8 and R9 form, with the nitrogen atom to which they are bonded, a cyclic amine chosen from the pyrrolyl, piperidyl, morpholinyl, pyrrolidinyl, azetidinyl and piperazinyl radicals optionally being substituted on the optional second nitrogen atom by an alkyl radical itself optionally substituted by one or more identical or different radicals chosen from halogen atoms and hydroxyl radicals;
the said products of formula (I) being in all the possible isomeric forms, racemic, enantiomeric and diastereoisomeric, and also the addition salts with inorganic and organic acids of the said products of formula (I).

In particular, the ring including Y can be composed of 4 to 7 ring members and can be saturated with Y representing an oxygen atom O, a sulphur atom S, optionally oxidized by one or two oxygen atoms, or a radical chosen from N—R7, CH—NH₂, CH—NHalk or CH—N(alk)₂, with R7 as defined above or below.

The present invention has in particular as subject-matter the products of formula (I) as defined above or below corresponding to the formula (IA) in which:

Bicyclic compound represents an indolinyl, 2-oxoindolinyl, indolyl or benzimidazole radical bearing the R2, R3 and R4 radicals,
R has the meaning indicated above or below,
R2, R3 and R4, which are identical or different, represent a hydrogen atom, a halogen atom, CN or an alkyl or alkoxy radical optionally substituted by one or more fluorine atoms;
R5 represents a hydrogen atom or a fluorine or chlorine atom;
Z represents SO₂ or CO;
D represents a hydrogen atom or a cyclopropyl, methyl, ethyl, propyl or butyl radical optionally substituted by one or more identical or different radicals chosen from the fluorine atom and the hydroxyl, amino, alkylamino, dialkylamino, piperidinyl, morpholinyl, azetidinyl, piperazinyl, pyrrolidinyl and pyrrolyl radicals;
the ring (Y) is chosen from the cyclohexyl radical, itself optionally substituted by amino; the tetrahydropyran radical; the dioxidothienyl radical; and the pyrrolidinyl, piperidinyl and azepinyl radicals optionally substituted on their nitrogen atom by a radical chosen from the methyl, propyl, butyl, isopropyl, isobutyl, isopentyl or ethyl radicals, themselves optionally substituted by one or more radicals chosen from halogen atoms, hydroxyl radicals and phenyl, quinolyl, pyridyl optionally oxidized on its nitrogen atom, thienyl, thiazolyl, thiadiazolyl, tetrazolyl, pyrazinyl, furyl and imidazolyl radicals, the latter cyclic radicals being themselves optionally substituted by one or more identical or different radicals chosen from halogen atoms and hydroxyl, methyl and methoxy radicals;
the said products of formula (I) being in all the possible isomeric forms, racemic, enantiomeric and diastereoisomeric, and also the addition salts with inorganic and organic acids of the said products of formula (I).

The present invention has in particular as subject-matter the products of formula (I) as defined above or below corresponding to the formula (IA) in which:

Bicyclic compound represents an indolinyl, 2-oxoindolinyl, indolyl or benzimidazolyl radical bearing the R2, R3 and R4 radicals,
R has the meaning indicated above or below,
R2, R3 and R4, which are identical or different, represent a hydrogen atom, a halogen atom, CF₃, CN, or a methyl or methoxy radical;
R5 represents a hydrogen atom;
D represents a methyl radical or an ethyl radical optionally substituted by an amino, alkylamino, dialkylamino or pyrrolidinyl radical;
the ring including Y represents a cyclohexyl radical itself optionally substituted by amino or a piperidinyl radical optionally substituted on its nitrogen atom by a methyl, propyl, butyl, isopropyl, isobutyl, isopentyl or ethyl radical, themselves optionally substituted by one or more halogen atoms or a radical chosen from hydroxyl; thiadiazolyl; tetrazolyl; phenyl, itself optionally substituted by halogen; quinolyl; pyridyl, optionally oxidized on its nitrogen atom; furyl; and imidazolyl, itself optionally substituted by alkyl;
the said products of formula (I) being in all the possible isomeric forms, racemic, enantiomeric and diastereoisomeric, and also the addition salts with inorganic and organic acids of the said products of formula (I).

Mention is thus particularly made of the products of formula (I) in which R5 represents a hydrogen atom, the other substituents R, R1, R2, R3, R4, and ring(Y) of the said products of formula (I) being chosen from the values indicated above.

When NR8R9 does not form a cyclic amine, then NR8R9, in particular, is such that R8 represents a hydrogen atom or an alkyl radical and R9 is chosen from all the values defined for R8.

The radical NR8R9 may also represent the values defined above for NRaRb.

When one of R2, R3 and R4 represents alkoxy, methoxy is preferred.

One subject of the present invention is especially the products of formula (I) such as defined above or below corresponding to the formula (IA) in which:

Bicyclic compound represents an indolinyl, 2-oxoindolinyl, indolyl or benzimidazolyl radical bearing the R2, R3 and R4 radicals,
R has the meaning indicated above or below,
R2, R3 and R4, which are identical or different, represent a hydrogen atom, a fluorine atom, CF₃, CN or a methyl or methoxy radical;
R5 represents a hydrogen atom;
D represents a hydrogen atom or a methyl radical or an ethyl radical optionally substituted by NH₂;
the ring (Y) is chosen from the tetrahydropyranyl or dioxidothienyl radicals and the pyrrolidinyl, piperidinyl and azepinyl radicals optionally substituted on their nitrogen atoms (in the 2 or 3 position of the ring) by a methyl, ethyl, propyl or butyl radical, themselves optionally substituted by one or more halogen atoms or a phenyl, pyridyl, thienyl, thiazolyl, thiadiazolyl, pyrazinyl, furyl or imidazolyl radical;
the said products of formula (I) being in all the possible isomeric forms, racemic, enantiomeric and diastereoisomeric, and also the addition salts with inorganic and organic acids of the said products of formula (I).

A subject of the present invention is in particular the products of formula (I) as defined above or below corresponding to the formula (IB) in which bicyclic compound, R, R2, R3, R4, R5 and Z have the meanings indicated above or below and the ring(N) represents one of the rings defined below:
an azetidinyl or pyrrolidinyl ring substituted at position 3 by R1 and R6 such as defined above or below;
a piperidinyl and azepinyl ring substituted at position 3 or 4 by R1 and R6 such as defined above or below;
an 8-azabicyclo[3,2,1]octan-3-yl, 6-azabicyclo[3.2.1]octan-3-yl or 3-azabicyclo[3.2.1]octan-8-yl) ring;
   the said products of formula (I) being in all the possible isomeric forms, racemic, enantiomeric and diastereoisomeric, and also the addition salts with inorganic and organic acids of the said products of formula (I).

A subject of the present invention is in particular the products of formula (I) as defined above or below corresponding to the formula (IB) in which bicyclic compound, R, R2, R3, R4, R5 and Z have the meanings indicated above or below and the ring(N) represents a pyrrolidinyl ring substituted at position 3 by R1 and R6 such as defined above or below or a piperidinyl ring substituted at position 3 or 4 by R1 and R6 such as defined above or below,

the said products of formula (I) being in all the possible isomeric forms, racemic, enantiomeric and diastereoisomeric, and also the addition salts with inorganic and organic acids of the said products of formula (I).

A subject of the present invention is in particular the products of formula (I) as defined above or below corresponding to the formula (IB) in which:

Bicyclic compound represents an indolinyl, 2-oxoindolinyl, indolyl or benzimidazolyl radical bearing the R2, R3 and R4 radicals,
R has the meaning indicated above or below,
R2, R3 and R4, which are identical or different, represent a hydrogen atom, a halogen atom, CN or a alkyl or alkoxy radical optionally substituted by one or more halogen atoms;
R5 represents a hydrogen atom or a halogen atom;
Z represents CO or SO₂;
the ring(N) i.e.

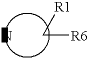

represents a pyrrolidinyl radical substituted at position 3 by R1 and R6 or a piperidinyl ring substituted at position 3 or 4 by R1 and R6, being understood that R1 and R6 represent one of the 5 following alternatives i) to v):
i) R1 represents -X1-R7 where X1 represents —CH₂ and R7 represents a heterocycloalkyl, phenyl or heteroaryl ring, all optionally substituted;
and R6 represents a hydrogen atom, or hydroxyl, —CH₂OH, —CO—NRaRb, and —CO₂Et radicals;
ii) R1 represents -X2-R7 where X2 represents:

### —O—; —CH(OH)—; —CH(OH)—CH₂—, —CO—;

### —CH(NRaRb)—; —C═NOH—; —C═N—NH₂— and

—(CH2)ₙ₁-NRc-(CH₂)ₙ₂—; and R7 represents a heterocycloalkyl, phenyl or heteroaryl ring, all optionally substituted;
and R6 represents hydrogen;
iii) R1 represents —NRc-A where A represents a hydrogen atom or an alkyl radical containing 1 to 4 carbon atoms that is linear or branched optionally substituted by a radical chosen from —PO(OEt)₂, —OH, —OEt, —CF₃, —CO—NR8R9 and SO₂-alk; and R6 represents hydrogen;
being understood that when A represents a hydrogen atom then z represents CO;
iv) R1 represents —CH₂—NRc-A where A represents a hydrogen atom or an alkyl radical containing 1 to 4 carbon atoms that is linear or branched starting from 3 carbon atoms and optionally substituted by a radical SO₂-alk; and R6 represents hydrogen;
v) R1 represents —CO—N(Rc)-OR′c and R6 represents hydrogen;
where n, n1 and n2, being identical or different, represent an integer from 0 to 2;
Rc and R′c, being identical or different, represent a hydrogen atom or an alkyl radical containing 1 to 2 carbon atoms;
NRaRb is such that either Ra and Rb, being identical or different, represent a hydrogen atom or an alkyl radical containing from 1 to 4 carbon atoms optionally substituted by one or more identical or different radicals chosen from halogen atoms and hydroxyl, alkoxy, NH₂, NHalkyl and N(alkyl)₂ radicals; or Ra and Rb form, with the nitrogen atom to which they are bonded, a morpholinyl or pyrrolidinyl radical optionally substituted by one or more identical or different radicals chosen from halogen atoms and alkyl radicals themselves optionally substituted by one or more halogen atoms; all the heterocycloalkyl, phenyl and heteroaryl radicals being optionally substituted by one or more radicals, identical or different, chosen from halogen atoms; hydroxyl; cyano; NR8R9 radicals; and alkyl, cycloalkyl, alkoxy, phenyl, heterocycloalkyl and heteroaryl radicals, themselves optionally substituted by one or more identical or different radicals chosen from halogen atoms and hydroxyl, alkoxy, OCF₃, CH₃, —CH₂OH, CN, CF₃, OCF₃ or NRaRb radicals;
NR8R9 is such that either R8 and R9, being identical or different, are such that R8 represents a hydrogen atom, a linear or branched alkyl radical containing at most 4 carbon atoms or a cycloalkyl radical containing from 3 to 6 ring members, the alkyl and cycloalkyl radicals being themselves optionally substituted by one or more halogen atoms or a hydroxyl radical; and R9 represents a hydrogen atom or an alkyl radical optionally substituted by one or more identical or different radicals chosen from halogen atoms and hydroxyl, alkoxy, NH₂, NHalkyl, N(alkyl)₂, phenyl, heterocycloalkyl or heteroaryl radicals themselves optionally substituted by one or more radicals chosen from halogen atoms and hydroxyl, OCH₃, CH₃, —CH₂OH, CN, CF₃, OCF₃, NH₂, NHalk or N(alk)₂ radicals; or R8 and R9 form, with the nitrogen atom to which they are bonded, a cyclic amine chosen from pyrrolyl, piperidyl, morpholinyl, pyrrolidinyl, azetidinyl and piperazinyl optionally substituted by one or more alkyl radicals themselves optionally substituted by one or more halogen atoms;
the said products of formula (I) being in all the possible isomeric forms, racemic, enantiomeric and diastereoisomeric, and also the addition salts with inorganic and organic acids of the said products of formula (I).

In the products of formula (I) corresponding to the formula (IB) such as defined above, all the heterocycloalkyl, phenyl and heteroaryl radicals that R7 represents may especially be optionally substituted by one or more identical or different radicals chosen from halogen atoms; NR8R9 radicals; and alkyl, cycloalkyl, alkoxy, phenyl, heterocycloalkyl and heteroaryl radicals, themselves optionally substituted by one or more identical or different radicals chosen from halogen atoms and hydroxyl, alkoxy, OCF₃, CH₃, —CH₂OH, CN, CF₃, OCF₃, NH₂, NHalk, N(alk)₂, pyrrolidinyl, piperidinyl or morpholinyl radicals optionally substituted by one or more identical or different radicals chosen from halogen atoms and alkyl radicals themselves optionally substituted by one or more halogen atoms.

A subject of the present invention is in particular the products of formula (I) as defined above or below corresponding to the formula (IB) in which bicyclic compound, R, R2, R3, R4, R5, Z and the ring(N) have the meanings indicated above or below and R1 and R6 are such that:

either R1 represents -X-R7 where X1 represents —CH₂— and R6 represents a hydrogen atom or the hydroxyl, CH₂—OH; —CO—N(CH₃)₂, —CO—NHCH₃, —CO—NH—(CH₂)₂—N(CH₃)₂ and —CO₂Et radicals;
or R1 represents -X2-R7 where X represents:
—O—, —CHOH—, —CH(OH)—CH₂—, —CO—, —CHNH₂—, —NH—CH₂—, —N(CH₃)—CH₂— and CH₂—NH—CH₂—; and R6 represents hydrogen;
and R7 is chosen from the pyrrolidinyl, piperidinyl, piperazinyl, pyrimidinyl, morpholinyl, thiomorpholinyl, tetrahydrofuran, hexahydrofuranyl, phenyl, pyridyl, thienyl, thiazolyl, dithiazolyl, pyrazolyl, pyrazinyl, furyl, imidazolyl, pyrrolyl, oxazolyl, isoxazolyl, benzofuranyl, benzodihydrofuranyl, benzoxadiazolyl, benzothiodiazolyl, benzothienyl, quinolyl, isoquinolyl radicals,
all these radicals that R7 represents being optionally substituted by one or more identical or different radicals chosen from halogen atoms and hydroxyl, methyl, methoxy, hydroxymethyl, alkoxymethyl, cyano, NH₂, NHalk, N(alk)₂, —CH₂—NH₂, —CH₂—NHalk, —CH₂—N(alk)₂, phenyl, morpholinyl and CH₂-morpholinyl radicals themselves optionally substituted by one or more identical or different radicals chosen from halogen atoms and hydroxyl, CH₃, OCH₃, —CH₂OH, CN, CF₃, OCF₃, NH₂, NHalk or N(alk)₂ radicals;
the said products of formula (I) being in all the possible isomeric forms, racemic, enantiomeric and diastereoisomeric, and also the addition salts with inorganic and organic acids of the said products of formula (I).

A subject of the present invention is in particular the products of formula (I) as defined above or below corresponding to the formula (IB) in which bicyclic compound, R, R2, R3, R4, R5, Z and the ring(N) have the meanings indicated above or below and R1 and R6 are such that: either R1 represents -X1-R7 where X1 represents —CH₂— and R6 represents a hydrogen atom or the hydroxyl, CH₂—OH; —CO—N(CH₃)₂, —CO—NHCH₃, —CO—NH—(CH₂)₂—N(CH₃)₂ and —CO₂Et radicals; or R1 represents -X2-R7 where X2 represents:

—O—, —CHOH—, —CH(OH)—CH₂—, —CO—, —CHNH₂—, —NH—CH₂—, —N(CH₃)—CH₂— and CH₂—NH—CH₂—; and R6 represents hydrogen;
and R7 is chosen from pyrrolidinyl, piperidinyl, piperazinyl, pyrimidinyl, morpholinyl, thiomorpholinyl, tetrahydrofuranyl, phenyl, pyridyl, thienyl, thiazolyl, dithiazolyl, pyrazolyl, pyrazinyl, furyl, imidazolyl, pyrrolyl, oxazolyl, isoxazolyl, benzodihydrofuranyl, benzoxadiazolyl, benzothiadiazolyl, benzothienyl, quinolyl, isoquinolyl radicals;
all these radicals that R7 represents being optionally substituted by one or more identical or different radicals chosen from halogen atoms and hydroxyl, methyl, methoxy, hydroxymethyl, alkoxymethyl, cyano, NH₂, NHalk, N(alk)₂, —CH₂—NH₂, —CH₂—NHalk, —CH₂—N(alk)₂, phenyl, morpholinyl and CH₂-morpholinyl radicals themselves optionally substituted by one or more identical or different radicals chosen from halogen atoms and hydroxyl, CH₃, OCH₃, —CH₂OH, CN, CF₃, OCF₃, NH₂, NHalk or N(alk)₂ radicals;
the said products of formula (I) being in all the possible isomeric forms, racemic, enantiomeric and diastereoisomeric, and also the addition salts with inorganic and organic acids of the said products of formula (I).

A subject of the present invention is in particular the products of formula (I) as defined above or below in which bicyclic compound, R, R1, R5, R6, Z, D, W, ring(Y) and ring(N) have the meanings indicated above or below;

R2, R3 and R4, being identical or different, represent a hydrogen atom, a halogen atom, CF₃, CN, or a methyl or methoxy radical;
and R5 represents a hydrogen atom;
the said products of formula (I) being in all the possible isomeric forms, racemic, enantiomeric and diastereoisomeric, and also the addition salts with inorganic and organic acids of the said products of formula (I).

A subject of the present invention is in particular the products of formula (I) as defined above or below in which bicyclic compound, R, R1, R6, Z, D, W, ring(Y) and ring(N) have the meanings indicated above or below and R2, R3 and R4, being identical or different, represent a hydrogen atom, a fluorine atom, CF₃, CN or a methyl or methoxy radical;

R5 represents a hydrogen atom;
the said products of formula (I) being in all the possible isomeric forms, racemic, enantiomeric and diastereoisomeric, and also the addition salts with inorganic and organic acids of the said products of formula (I).

A subject of the present invention is in particular the products of formula (I) as defined above or below in which bicyclic compound, R, R1, R2, R3, R4, R5, R6, W, D, ring(Y) and ring(N) have the meanings indicated above or below and Z represents SO₂, the said products of formula (I) being in all the possible isomeric forms, racemic, enantiomeric and diastereoisomeric, and also the addition salts with inorganic and organic acids of the said products of formula (I).

A subject of the present invention is in particular the products of formula (I) as defined above or below in which bicyclic compound, R, R1, R2, R3, R4, R5, R6, W, D, ring(Y) and ring(N) have the meanings indicated above or below and Z represents CO, the said products of formula (I) being in all the possible isomeric forms, racemic, enantiomeric and diastereoisomeric, and also the addition salts with inorganic and organic acids of the said products of formula (I).

A subject of the present invention is in particular the products of formula (I) as defined above or below corresponding to the following names:
4-[4-(5-fluoro-2,3-dihydroindol-1-yl)pyrimidin-2-ylamino]-N-[1-(1-methyl-1H-pyrrol-2-ylmethyl)piperidin-4-yl]-N-(2-pyrrolidin-1-ylethyl)benzenesulphonamide;
4-[4-(5-fluoro-2,3-dihydroindol-1-yl)pyrimidin-2-ylamino]-N-(2-pyrrolidin-1-ylethyl)-N-(tetrahydropyran-4-yl)benzenesulphonamide;
4-[4-(5-fluoro-2,3-dihydroindol-1-yl)pyrimidin-2-ylamino]-N-methyl-N-(1-methylpiperidin-4-yl)benzenesulphonamide;
4-{[4-(5-cyano-1H-indol-1-yl)pyrimidin-2-yl]amino}-N-piperidin-4-yl-N-(2-pyrrolidin-1-ylethyl)benzenesulphonamide;
4-{[4-(1H-benzimidazol-1-yl)pyrimidin-2-yl]amino}-N-piperidin-4-yl-N-(2-pyrrolidin-1-ylethyl)benzenesulphonamide;
   the said products of formula (I) being in all the possible isomeric forms, racemic, enantiomeric and diastereoisomeric, and also the addition salts with inorganic and organic acids of the said products of formula (I).

A subject of the present invention is also the methods of preparation of the products of formula (I) as defined above by using methods known to a person skilled in the art.

A subject of the present invention is in particular the method of preparation of the products of formula (I) as defined above characterized in that a product of formula (II):

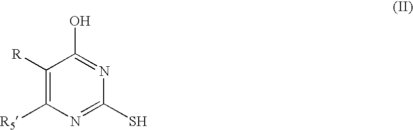

in which R₅′ has the meaning indicated above for R5 in which the optional reactive functional groups are optionally protected,
is converted to a product of formula (III):

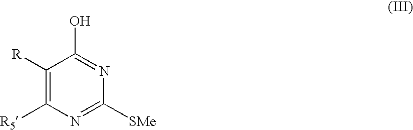

in which R₅′ has the meaning indicated above,
which product of formula (III) is reacted with the aniline of formula (IV):

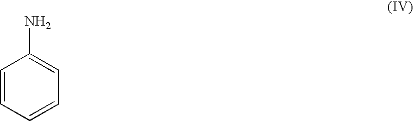

to obtain a product of formula (V):

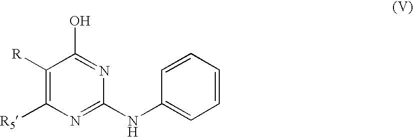

in which R₅′ has the meaning indicated above,
which product of formula (V) is converted to a product of formula (VI):

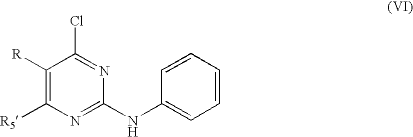

in which R₅′ has the meaning indicated above,
route a) (z=SO₂) product of formula (VI) which is reacted with chlorosulphonic acid SO₂(OH)Cl to obtain the corresponding product of formula (VII):

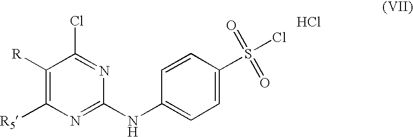

in which R₅′ has the meaning indicated above,
product of formula (VII) which is reacted
either with an amine of formula (VIII)1:

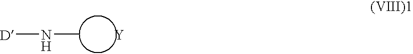

in which D′ has the meaning indicated above for D in which the optional reactive functional groups are optionally protected by protective groups and Y has the meaning indicated above,
to obtain a product of formula (IX) A1:

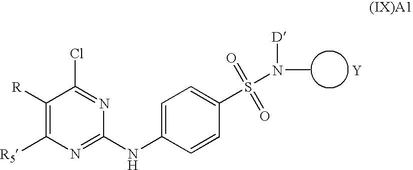

in which R₅′, D′ and Y have the meanings indicated above,
or with an amine of formula (VIII)2:

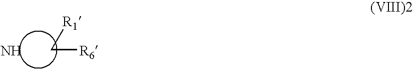

in which R₁′ and R₆′ have the meanings indicated above respectively for R1 and R6, in which the optional reactive functional groups are optionally protected by protective groups,
to obtain a product of formula (IX) A2:

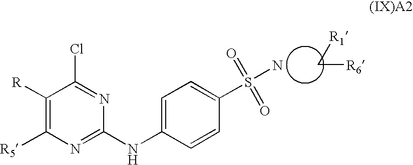

in which R₁′, R₅′ and R₆′ have the meanings indicated above,
which product of formula (IX) A1 or (IX) A2 is reacted with a nitroheterocycle of formula (X):

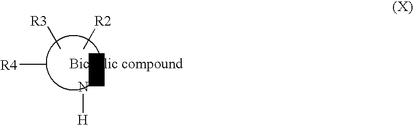

to respectively obtain a product of formula (IA)1

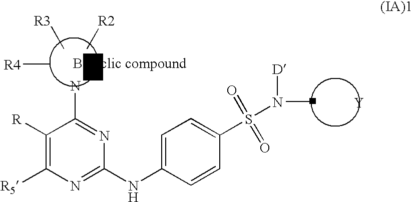

in which R2′, R3′, R4′, R5′, D′ and Y have the meanings indicated above,
or a product of formula (IA)2:

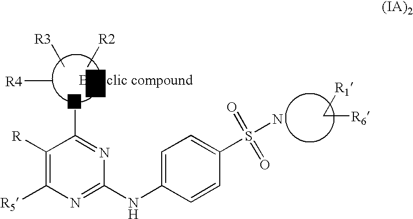

in which R1′, R2, R3, R4, R₅′ and R6′ have the meanings indicated above,
route b) product of formula (III) such as defined above that is reacted with the methyl ester of 4-aminobenzoic acid to obtain the product of formula (XI):

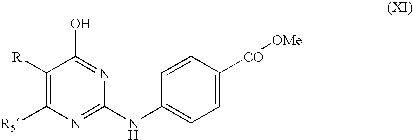

in which R₅′ has the meaning indicated above,
which product of formula (XI) is reacted with a nitroheterocycle of formula (X) such as defined above to obtain a product of formula (XII):

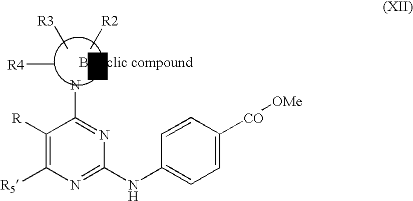

in which R2, R3, R4 and R₅′ have the meanings indicated above,
which product of formula (XII) is converted to its corresponding acid of formula (XIII):

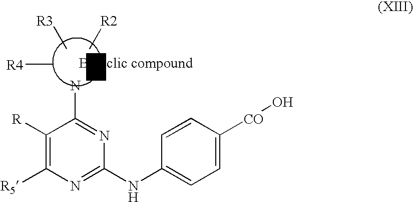

in which R2, R3, R4 and R₅′ have the meanings indicated above,
which product of formula (XIII) is reacted:
either with an amine of formula (VIII)1 such as defined above to obtain a product of formula (IB)1:

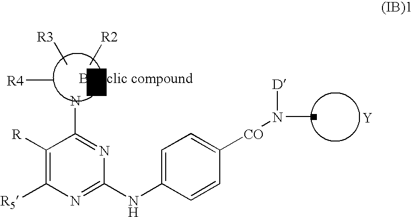

in which R2, R3, R4, R₅′, D′ and Y have the meanings indicated above,
or with an amine of formula (VIII)2 such as defined above to obtain a product of formula (IB)2:

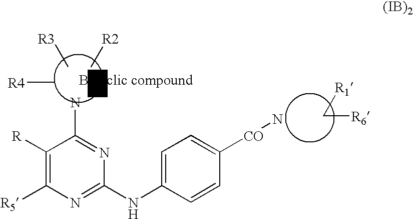

in which R₁′, R2, R3, R4, R₅′ and R₆′ have the meanings indicated above,
which products of formula (IA)1, (IA)2, (IB)1 and (IB)2 can be products of formula (I) in which z represents SO2 or CO respectively, and which, in order to obtain products or other products of formula (I), can be subjected, if desired and if necessary, to one or more of the following conversion reactions, in any order:
a) an oxidation reaction on an alkylthio group to give the corresponding sulphoxide or sulphone,
b) a conversion reaction on an alkoxy functional group to give a hydroxyl functional group or also on a hydroxyl functional group to give an alkoxy functional group,
c) an oxidation reaction on an alcohol functional group to give an aldehyde or ketone functional group,
d) an elimination reaction on the protective groups which can be carried by the protected reactive functional groups,
e) a salification reaction by an inorganic or organic acid in order to obtain the corresponding salt,
f) a resolution reaction on the racemic forms to give resolved products,
the said products of formula (I) thus obtained being in all the possible isomeric forms, racemic, enantiomeric and diastereoisomeric.

A subject of the present invention is also a method of preparation of the products of formula (I) as defined above corresponding to the formula (IA) such as defined above in which Y represents the NR10 radical as defined above with R10 representing CH₂—RZ and RZ representing an alkyl, alkenyl or alkynyl radical, all optionally substituted by a naphthyl radical or by one or more identical or different radicals chosen from halogen atoms and phenyl and heteroaryl radicals, all these naphthyl, phenyl and heteroaryl radicals being themselves optionally substituted by one or more identical or different radicals chosen from halogen atoms and the hydroxyl, alkoxy, alkyl, hydroxyalkyl, alkoxyalkyl, CF₃, NH₂, NHalk or N(alk)₂ radicals,

method characterized in that the compound of formula (XIV):

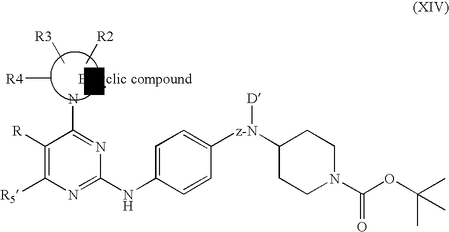

in which R2, R3, R4 and R₅′ have the meanings indicated above and z represents SO₂ or CO,
is subjected to a reaction for deprotecting the carbamate functional group in order to obtain a product of formula (XV):

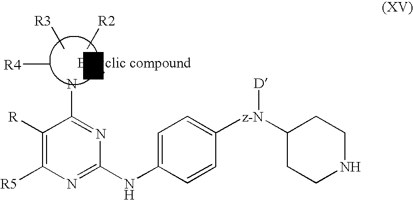

in which R2, R3, R4 and R5 have the meanings indicated above, and D′ has the meaning indicated above for D in which the optional reactive functional groups are optionally protected by protective groups,
which product of formula (XV) is subjected to reducing amination conditions in the presence of an aldehyde or ketone of formula (XVI):

RZ′—CR8′O (XVI)

in which RZ′ and R8′ have the meanings indicated above for RZ and R8 respectively, in which the optional reactive functional groups are optionally protected by protective groups,
to obtain a product of formula (IA):

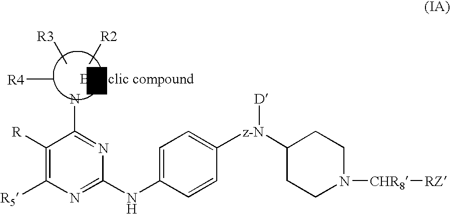

in which R2, R3, R4, R₅′, z, D′, R₈′ and RZ′ have the meanings indicated above,
which products of formula (IA) can be products of formula (I) and which, in order to obtain products or other products of formula (I), can be subjected, if desired and if necessary, in any order, to one or more of the conversion reactions a) to f) as defined above,
the said products of formula (I) thus obtained being in all the possible isomeric forms, racemic, enantiomeric and diastereoisomeric.

In preferred conditions for implementation of the invention, the methods described above may be carried out in the following way:

The product of formula (II) is converted to a product of formula (III) such as defined above, in particular in water in the presence of sodium hydroxide and methyl iodide at ambient temperature.

The product of formula (III) thus obtained is subjected to the action of the aniline of formula (IV) such as defined above, in particular in an alcohol such as, for example, butanol or dimethylformamide, in the presence or absence of a strong acid (HCl) in a catalytic amount under reflux conditions in order to obtain a product of formula (V) such as defined above.

The product of formula (V) such as defined above is converted to a product of formula (VI) by the action of phosphorus oxychloride POCl₃ between 90 and 110° C. for one to two hours.

According to the route a) defined above, the product of formula (VI) is subjected to the action of chlorosulphonic acid, in particular first at 0° C. then at ambient temperature to obtain a product of formula (VII) such as defined above.

The product of formula (VII) thus obtained is subjected to the action of an amine of formula (VIII)1 or (VIII)2 such as defined above, in particular in dichloromethane or a dichloromethane/THF mixture or dimethylformamide at ambient temperature, in the presence of an organic base such as triethylamine, diisopropylethylamine or N-methylmorpholine, to respectively obtain a product of formula (IX) A1 or (IX) A2 such as defined above.

The product of formula (IX) A1 or (IX) A2 is reacted with a nitroheterocycle of formula (X) such as defined above in a solvent such as toluene, dioxane, dimethylformamide, at temperatures between 100 and 150° C., with, optionally, alkaline agents of the type: K₂CO₃, Na₂CO₃, Cs₂CO₃, caesium fluoride CsF to thus respectively obtain a product of formula (IA)1 or (IA)2 such as defined above.

According to route b) defined above, the product of formula (III) such as defined above is subjected to the action of the methyl ester of 4-aminobenzoic acid, in particular in an alcohol such as butanol at a temperature of 100 to 140° C., to give the product of formula (XI) such as defined above.

The product of formula (XI) is reacted with a nitroheterocycle of formula (X) such as defined above under the conditions defined above for obtaining a product of formula (XII).

This product of formula (XII) is saponified to give its corresponding acid of formula (XIII) while proceeding according to the normal methods known to a person skilled in the art, such as, in particular, by the action of sodium hydroxide or potassium hydroxide in water.

The product of formula (XIII) thus obtained is reacted with an amine of formula (VIII)1 or (VIII)2 such as defined above according to the coupling methods known to a person skilled in the art, such as, for example, by amide coupling in the presence of a coupling agent, such as BOP, DCC or TBTU, in a solvent, such as, for example, dimethylformamide or dichloromethane, in order to obtain respectively a product of formula (IB)1 or (IB)2 such as defined above.

The deprotection reaction on the carbamate functional group of the compound of formula (XIV) in order to obtain a product of formula (XV) can be carried out using, for example, an acid agent, such as pure trifluoroacetic acid at a temperature of approximately 0° C. or a mixture of this acid with an appropriate solvent, such as methylene chloride, at approximately 0° C., or also using hydrochloric acid in solution in ether or dioxane at a temperature of between 0° C. and ambient temperature.

The product of formula (XV) is subjected to reductive amination conditions in the presence of the aldehyde or ketone of formula (XVI), in order to obtain a product of formula (IA) as defined above, for example with sodium borocyanide or sodium triacetoxyborohydride, in a solvent, such as methanol, tetrahydrofuran (THF) or their mixture, as a medium with a pH between 4 and 7.

Depending on the values of R₁′, R2′, R3′, R4′, R5′, R6′, R8′, D′ and RZ′, the products of formulae (IA)1, (IA)2, (IB)1 and (IB)2 as defined above can thus constitute products of formula (I) as defined above or can be converted to products of formula (I) by the usual methods known to a person skilled in the art, for example by being subjected to one or more of the reactions a) to f) indicated above.

Furthermore, it may be noted that such reactions a) to f) for the conversion of substituents into other substituents can also be carried out on the starting materials and on the intermediates as defined above before continuing the synthesis according to the reactions indicated in the above processes.

The various reactive functional groups which may be carried by some compounds of the reactions defined above can, if necessary, be protected: this concerns, for example, hydroxyl, amino and monoalkylamino radicals, which can be protected by appropriate protective groups.

The following non-exhaustive list of examples of the protection of reactive functional groups may be mentioned:
hydroxyl groups can be protected, for example, by alkyl radicals, such as tert-butyl, trimethylsilyl, tert-butyldimethylsilyl, methoxymethyl, tetrahydropyranyl, benzyl or acetyl,
amino groups can be protected, for example, by acetyl, trityl, benzyl, tert-butoxycarbonyl, benzyloxycarbonyl or phthalimido radicals or other radicals known in the chemistry of peptides and can then be released under the usual conditions known to a person skilled in the art.

The reactions to which the products of formula (I′) as defined above can be subjected, if desired or if necessary, can be carried out, for example, as indicated below.

The saponification reactions can be carried out according to the usual methods known to a person skilled in the art, such as, for example, in a solvent, such as methanol or ethanol, dioxane or dimethoxyethane, in the presence of sodium hydroxide or potassium hydroxide.

The reduction or oxidation reactions can be carried out according to the usual methods known to a person skilled in the art, such as, for example, in a solvent, such as ethyl ether or tetrahydrofuran, in the presence of sodium borohydride or lithium aluminum hydride; or, for example, in a solvent, such as acetone or tetrahydrofuran, in the presence of potassium permanganate or pyridinium chlorochromate.

a) the possible alkylthio groups of the products described above can, if desired, be converted to the corresponding sulphoxide or sulphone functional groups under the usual conditions known to a person skilled in the art, such as, for example, with peracids, such as, for example, peracetic acid or meta-chloroperbenzoic acid, or also with oxone, sodium periodate, in a solvent, such as, for example, methylene chloride or dioxane, at ambient temperature.

The production of the sulphoxide functional group can be promoted by an equimolar mixture of the product including an alkylthio group and of the reactant, such as, in particular, a peracid.

The production of the sulphone functional group can be promoted by a mixture of the product including an alkylthio group with an excess of the reactant, such as, in particular, a peracid.

b) The possible alkoxy functional groups, such as, in particular, methoxy functional groups, of the products described above can, if desired, be converted to a hydroxyl functional group under the usual conditions known to a person skilled in the art, for example with boron tribromide, in a solvent, such as, for example, methylene chloride, with pyridine hydrobromide or hydrochloride or also with hydrobromic or hydrochloric acid in water or trifluoroacetic acid at reflux.
c) The possible alcohol functional groups of the products described above can, if desired, be converted to an aldehyde or ketone functional group by oxidation under the usual conditions known to a person skilled in the art, such as, for example, by the action of manganese oxide, in order to obtain aldehydes, or by the action of potassium permanganate or pyridinium chlorochromate, in order to access ketones.
d) The elimination of protective groups, such as, for example, those indicated above, can be carried out under the usual conditions known to a person skilled in the art, in particular by acid hydrolysis carried out with an acid, such as hydrochloric, benzenesulphonic, para-toluenesulphonic, formic or trifluoroacetic acid, or also by catalytic hydrogenation.

The phthalimido group can in particular be eliminated with hydrazine.

A list of various protective groups which can be used will be found, for example, in Patent BF 2 499 995.

e) The products described above can, if desired, form the subject of salification reactions, for example with an inorganic or organic acid, according to the usual methods known to a person skilled in the art.
f) The possible optically active forms of the products described above can be prepared by resolution of the racemates according to the usual methods known to a person skilled in the art.

Illustrations of such reactions defined above are given in the preparation of the examples described below.

The starting materials of formulae (II), (IV) and (VIII)1 and (VIII)2 may be known, may be obtained commercially or may be prepared according to the usual methods known to a person skilled in the art, in particular from commercial products, for example by subjecting them to one or more reactions known to a person skilled in the art, such as, for example, reactions described above in a) to f).

The products of formula (II), which are pyrimidine derivatives, can be commercial products, such as, for example, dichloropyrimidine and trichloropyrimidine.

The amines of formula (VIII)1 or (VIII)2 can also be commercially available, such as, for example, methyl(1-methylpiperidin-4-yl)amine.

The amines of formula (VIII)1 or (VIII)2 which are not commercially available can be prepared according to methods known to a person skilled in the art.

It may be indicated that, in order to obtain products of formula (I) corresponding to the formula (IA) as defined above in which R1, R2, R3, R4, R5, z and D have the meanings indicated above and ring(Y) is such that Y represents NR10 and includes a carbon bridge composed of 1 to 3 carbons, use may be made, as starting materials, of bicyclic amines which can be obtained from commercial compounds, such as tropinone or pseudopelletierine, according to the following references:
Tetrahedron, 2002, 58, 5669-5674
J. Org. Chem., 1996, 61, 3849-3862
J. Med. Chem., 1993, 36, 3703-3720
J. Chem. Soc. Perkin Trans. 1, 1991, 1375-1381
J. Med. Chem., 1994, 37, 2831-2840

Mention may be made, by way of examples, of the following compounds:
N,9-dimethyl-9-azabicyclo[3.3.1]nonan-3-amine

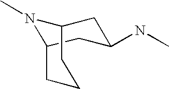

N,6-dimethyl-6-azabicyclo[3.2.1]octan-3-amine

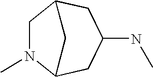

N,3-dimethyl-3-azabicyclo[3.2.1]octan-8-amine

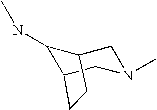

N,3-dimethyl-3-azabicyclo[3.3.1]nonan-9-amine

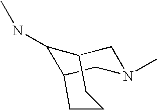

It can be pointed out that to obtain products of formula (I) corresponding to the formula (IB) such as defined above in which the ring(N) contains a carbon bridge composed of 1 to 3 carbons, it is possible to use bicyclic amines as starting materials which may be obtained from commercial compounds such as tropinone, the pseudo-pelletrivine according to the references below:
Tetrahedron 2002, 58, 5669-5674
J. Org. Chem. 1996, 61, 3849-3862
J. Med. Chem. 1993, 36, 3703-3720
J. Chem. Soc. Perkin Trans. 1, 1991, 1375-1381
J. Med. Chem. 1994, 37, 2831-2840

As examples of ring(N) mention may be made of the following compounds:
9-azabicyclo[3.3.1]nonan-3-amine

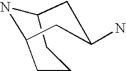

6-azabicyclo[3.2.1]octan-3-amine

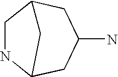

3-azabicyclo[3.2.1]octan-8-amine

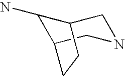

3-azabicyclo[3.3.1]nonan-9-amine

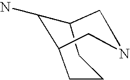

These bicyclic compounds may be bonded to z by their intracyclic nitrogen or by their exocyclic nitrogen for suitable protection of the non-reactive nitrogen that is known to a person skilled in the art.

The heterocycles of formulae (X) are commercially available or else their synthesis is described in the chemical literature.

The present invention also relates to the method according to scheme 1 below, for preparing products of formula (I) corresponding to the formula (IB) such as defined above:

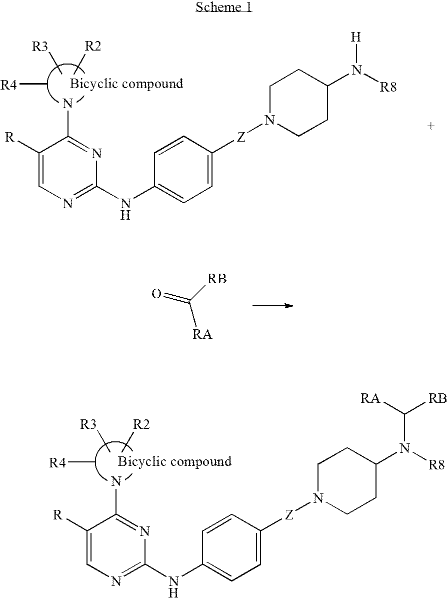

In such a Scheme 1, the NR8-CH(RA)(RB) radical represents certain values of NR8R9 as defined above with R8 as defined above and R9 representing —CH(RA)(RB), that is to say, as defined for R9, a linear or branched alkyl radical optionally substituted by one or more radicals chosen from halogen atoms and hydroxyl, alkoxy, NH₂, NHalkyl, N(alkyl)₂, alkylthio, phenyl and saturated or unsaturated heterocycle radicals, the phenyl and heterocycle radicals being themselves optionally substituted as indicated above.

In particular, RA can represent the hydrogen atom or CH₃ and RB can represent (CH₂)ₚ-G with G representing an optionally substituted heterocycle or phenyl radical as defined above and p representing an integer from 0 to 5.

Examples of aldehydes or ketones of formula (XVI) are given in the experimental section by way of non-limiting examples.

The stages of the synthetic process of Scheme 1 above can be carried out according to the usual methods known to a person skilled in the art.

The present invention also relates to the process according to Scheme 2 below for the preparation of products of formula (I) as defined above in which z represents CO:

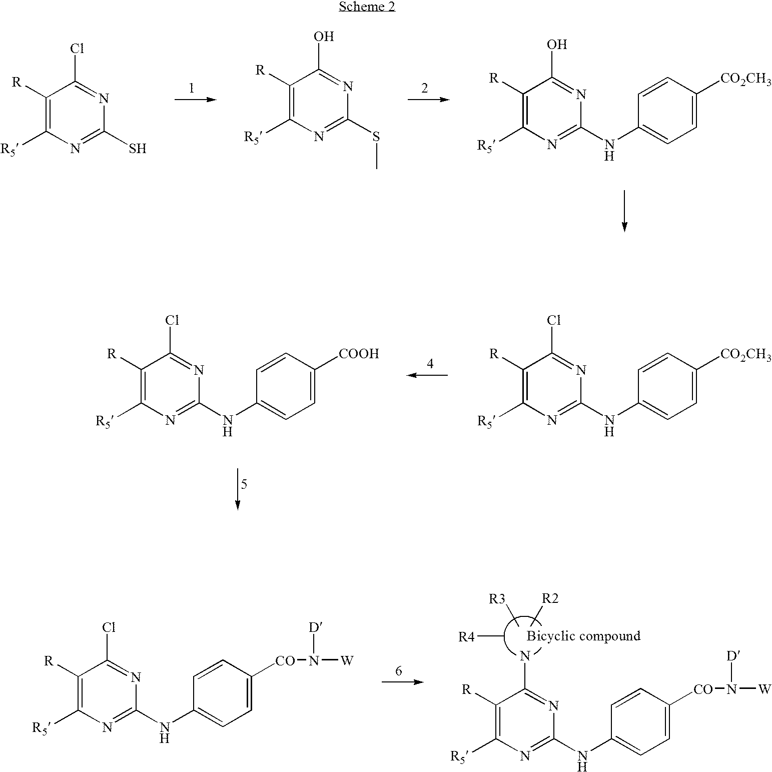

In such a Scheme 2, R2, R3, R4, R5′, D′ and W have the meanings indicated above.

The stages of the synthetic process of Scheme 2 above can be carried out by using the methyl ester of the aniline in stage 2 and the bicyclic compound substituted by R₂, R₃ or R₄ in stage 6 and by making use of the usual methods known to a person skilled in the art or as described in the present invention.

The experimental part below gives non-limiting examples of the preparation of products of formula (I) according to the present invention and also non-limiting examples of starting materials used in these preparations.

Finally, the present invention has as subject-matter some compounds of formulae (XIV), (XV), (IX) A1, (IX) A2, (XII) and (XIII) as novel industrial products.

The products of formula (I) as defined above and their addition salts with acids exhibit advantageous pharmacological properties.

The compounds of the present invention can thus inhibit the activity of kinases, in particular IKK1 and IKK2, with an IC₅₀ of less than 10 μM.

The compounds of the present invention can thus inhibit the activation of NF-KB and the production of cytokines with IC₅₀ values of less than 10 μM.

The compounds of the present invention can thus inhibit the proliferation of a large sample group of tumor cells with IC₅₀ values of less than 10 μM.

The compounds of the formula (I) can thus have a medicament activity, in particular as inhibitors of IKK1 and IKK2, and can be used in the prevention or treatment of diseases in which inhibition of IKK1 or IKK2 is beneficial, for example the prevention or treatment of diseases such as inflammatory diseases or diseases with an inflammatory component, such as, for example, inflammatory arthritis, including rheumatoid arthritis, osteoarthritis, spondylitis, Reiter's syndrome, psoriatic arthritis, bone resorption diseases; multiple sclerosis, inflammatory diseases of the intestines, including Crohn's disease; asthma, chronic obstructive pulmonary disease, emphysema, rhinitis, acquired myasthenia gravis, Graves' disease, graft rejection, psoriasis, dermatitis, allergic disorders, diseases of the immune system, cachexia, severe acute respiratory syndrome, septic shock, cardiac insufficiency, myocardial infarction, atherosclerosis, reperfusion injuries, AIDs, cancers and disorders characterized by resistance to insulin, such as diabetes, hyperglycaemia, hyperinsulinaemia, dyslipidaemia, obesity, polycystic ovarian disease, hypertension, cardiovascular disorders, syndrome X, autoimmune diseases, such as in particular systemic lupus, lupus erythematosus, glomerulonephritis induced by deficiencies in the immune system, autoimmune insulin-dependent diabetes, retinitis pigmentosa, aspirin-sensitive rhinosinusitis.

The products of formula (I) according to the present invention as modulators of apoptosis can be of use in the treatment of various human diseases including aberrations in apoptosis, such as cancers: such as in particular but without implied limitation follicular lymphomas, carcinomas with p53 mutations, hormone-dependent tumors of the breast, prostate and ovary, and precancerous lesions, such as familial adenomatous polyposis, viral infections (such as in particular but without implied limitation those caused by herpes virus, poxvirus, Epstein-Barr virus, Sindbis virus and adenovirus), myelodysplastic syndromes, ischemic disorders associated with myocardial infarction, cerebral congestion, arrhythmia, atherosclerosis, liver disorders induced by toxins or alcohol, hematological disorders, such as in particular but without implied limitation chronic anemia and aplastic anemia, degenerative diseases of the musculoskeletal system, such as in particular but without implied limitation osteoporosis, cystic fibrosis, diseases of the kidneys and cancers.

It thus appears that the compounds according to the invention have an anticancer activity and an activity in the treatment of other proliferative diseases, such as psoriasis, restenosis, atherosclerosis, AIDS, for example, and also in diseases caused by the proliferation of vascular smooth muscle cells of angiogenesis and then rheumatoid arthritis, neurofibromatosis, atherosclerosis, pulmonary fibrosis, restenosis following angioplasty or vascular surgery, the formation of hypertrophic scars, angiogenesis and endotoxic shock.

These medicaments are employed therapeutically, in particular in the treatment or prevention of diseases caused or exacerbated by the proliferation of cells and in particular tumor cells.

As inhibitor of the proliferation of tumor cells, these compounds are of use in the prevention and treatment of leukemia, solid tumors, both primary and metastatic, carcinomas and cancers, in particular: breast cancer, lung cancer, small intestine cancer, colon and rectal cancer, cancer of the respiratory tract, oropharynx and hypopharynx, esophageal cancer, liver cancer, stomach cancer, bile duct cancer, gall bladder cancer, pancreatic cancer, cancers of the urinary tract, including kidney, urothelium and bladder, cancers of the female genital tract, including cancer of the uterus, cervix or ovaries, choriocarcinoma and trophoblastomic cancer; cancers of the male genital tract, including cancer of the prostate, seminal vesicles or testicles, and germ cell tumors; cancers of the endocrine glands, including cancer of the thyroid, pituitary gland or adrenal glands; cancers of the skin, including hemangiomas, melanomas or sarcomas, including Kaposi's sarcoma; tumors of the brain, nerves, eyes or meninges, including astrocytomas, gliomas, glioblastomas, retinoblastomas, neurinomas, neuroblastomas, schwannomas or meningiomas; hematopoietic malignant tumors; leukemias, such as acute lymphocytic leukemia, acute myeloid leukemia, chronic myeloid leukemia, chronic lymphocytic leukemia, chloromas, plasmacytomas, T- or B-cell leukemias, non-Hodgkin's or Hodgkin's lymphomas, myelomas, various malignant haemopathies. The present invention has in particular as subject-matter the combinations defined as follows.

According to the present invention, the compound or compounds of formula (I) can be administered in combination with one (or more) anticancer active principle(s), in particular antitumor compounds, such as alkylating agents, such as alkylsulphonates (busulfan), dacarbazine, procarbazine, nitrogen mustards (chlormethine, melphalan, chlorambucil), cyclophosphamide or ifosfamide; nitrosoureas, such as carmustine, lomustine, semustine or streptozocin; antineoplastic alkaloids, such as vincristine or vinblastine; taxanes, such as paclitaxel or taxotere; antineoplastic antibiotics, such as actinomycin; intercalating agents, antineoplastic antimetabolites, folate antagonists or methotrexate; purine synthesis inhibitors; purine analogues, such as mercaptopurine or 6-thioguanine; pyrimidine synthesis inhibitors, aromatase inhibitors, capecitabine or pyrimidine analogues, such as fluorouracil, gemcitabine, cytarabine and cytosine arabinoside; brequinar; topoisomerase inhibitors, such as camptothecin or etoposide; anticancer hormonal agonists and antagonists, including tamoxifen; kinase inhibitors, imatinib; growth factor inhibitors; antiinflammatories, such as pentosan polysulphate, corticosteroids, prednisone or dexamethasone; antitopoisomerases, such as etoposide, anthracyclines, including doxorubicin, bleomycin, mitomycin and mithramycin; anticancer metal complexes, platinum complexes, cisplatin, carboplatin or oxaliplatin; interferon-alpha, triphenyl thiophosphoramide or altretamine; antiangiogenic agents; thalidomide; immunotherapy adjuvants; or vaccines.

According to the present invention, the compounds of formula (I) can also be administered in combination with one or more other active principles of use in one of the pathologies indicated above, for example an agent for combating vomiting, pain, inflammation or cachexia.

A subject-matter of the present invention is thus, as medicaments, the products of formula (I) as defined above and also the addition salts with pharmaceutically acceptable inorganic and organic acids of the said products of formula (I).

A subject-matter of the present invention is in particular, as medicaments, the products of formula (I) as defined above having the following names:
4-[4-(5-fluoro-2,3-dihydroindol-1-yl)pyrimidin-2-ylamino]-N-[1-(1-methyl-1H-pyrrol-2-ylmethyl)piperidin-4-yl]-N-(2-pyrrolidin-1-ylethyl)benzenesulphonamide;
4-[4-(5-fluoro-2,3-dihydroindol-1-yl)pyrimidin-2-ylamino]-N-(2-pyrrolidin-1-ylethyl)-N-(tetrahydropyran-4-yl)benzenesulphonamide;
4-[4-(5-fluoro-2,3-dihydroindol-1-yl)pyrimidin-2-ylamino]-N-methyl-N-(1-methylpiperidin-4-yl)benzenesulphonamide;
4-{[4-(5-cyano-1H-indol-1-yl)pyrimidin-2-yl]amino}-N-piperidin-4-yl-N-(2-pyrrolidin-1-ylethyl)benzenesulphonamide;
   and
4-{[4-(1H-benzimidazol-1-yl)pyrimidin-2-yl]amino}-N-piperidin-4-yl-N-(2-pyrrolidin-1-ylethyl)benzenesulphonamide,
   and also the addition salts with pharmaceutically acceptable inorganic and organic acids of the said products of formula (I).

Another subject-matter of the present invention is the pharmaceutical compositions comprising, as active principle, at least one of the products of formula (I) as defined above or a pharmaceutically acceptable salt of this product or a prodrug of this product and a pharmaceutically acceptable vehicle.

A subject-matter of the present invention is in particular the use of the products of formula (I) as defined above or of pharmaceutically acceptable salts of these products in the preparation of a medicament intended for the treatment or prevention of a disease by inhibition of the activity of the protein kinase IKK.

A subject-matter of the present invention is thus the use as defined above in which the protein kinase is in a mammal.

A subject-matter of the present invention is thus the use of a product of formula (I) as defined above in the preparation of a medicament intended for the treatment or prevention of a disease chosen from the diseases indicated above.

A subject-matter of the present invention is in particular the use of a product of formula (I) as defined above in the preparation of a medicament intended for the treatment or the prevention of a disease chosen from the following group: inflammatory diseases, diabetes and cancers.

A subject-matter of the present invention is in particular the use of a product of formula (I) as defined above in the preparation of a medicament intended for the treatment or prevention of inflammatory diseases.

A subject-matter of the present invention is in particular the use of a product of formula (I) as defined above in the preparation of a medicament intended for the treatment or prevention of diabetes.

A subject-matter of the present invention is in particular the use of a product of formula (I) as defined above in the preparation of a medicament intended for the treatment of cancers.

A subject-matter of the present invention is in particular the use of a product of formula (I) as defined above intended for the treatment of solid or nonsolid tumors.

A subject-matter of the present invention is in particular the use of a product of formula (I) as defined above intended for the treatment of cancers which are resistant to cytotoxic agents.

A subject-matter of the present invention is in particular the use of a product of formula (I) as defined above in the preparation of medicaments intended for cancer chemotherapy.

A subject-matter of the present invention is in particular the use of a product of formula (I) as defined above in the preparation of medicaments intended for cancer chemotherapy, alone or in combination or in the form of an association as defined above.

A subject-matter of the present invention is in particular the use of a product of formula (I) as defined above as IKK inhibitors.

The present invention relates very particularly to the products of formula (I) as defined above which constitute Examples 1 to 33 of the present invention.

The following examples illustrate the invention without, however, limiting it.

### EXPERIMENTAL PART

Products of formula (I) of the present invention are prepared according to scheme 3 below.

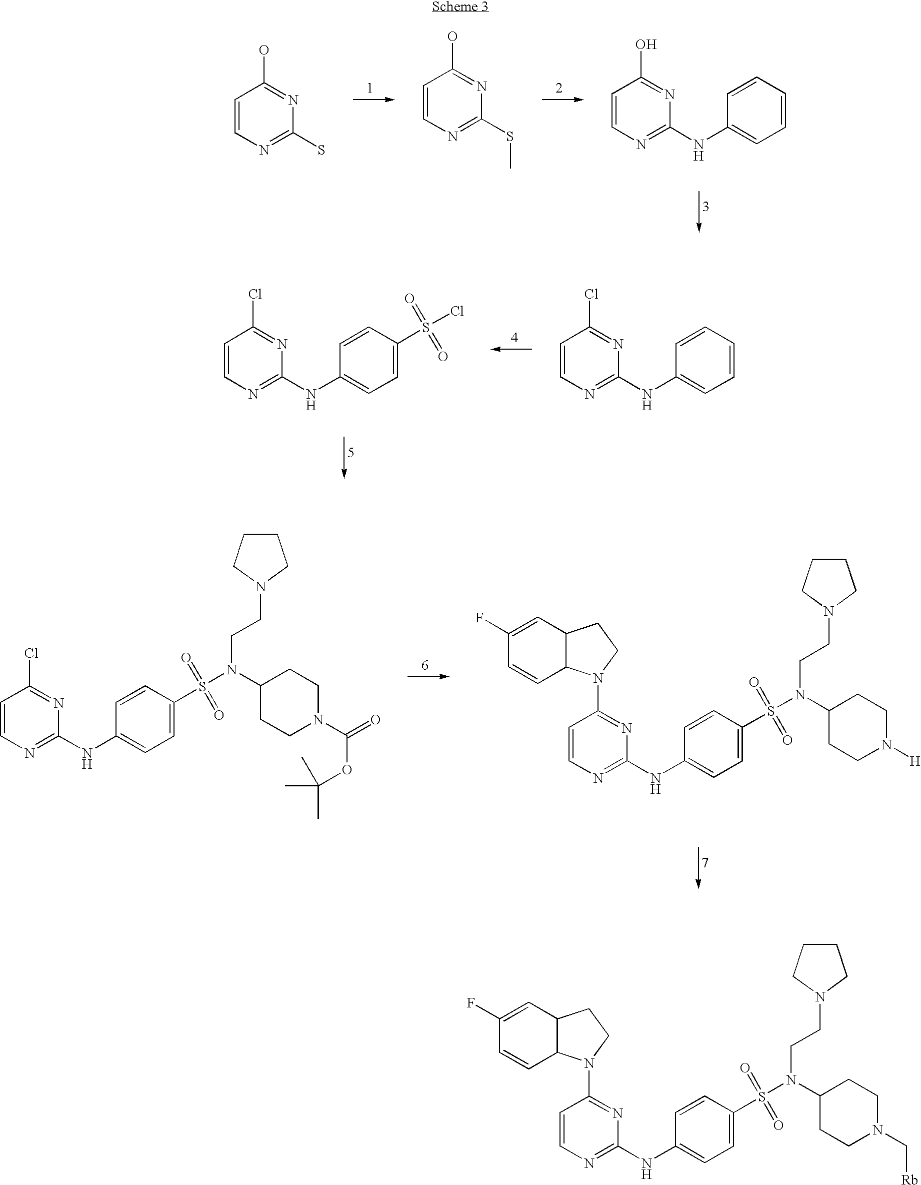

### Procedure 1a: Preparation of the hydrochloride of 2-[(2-chloropyrimidin-4-yl)amino]benzenesulphonyl chloride

### Stage 1: 2-(Methylthio)pyrimidin-4-ol

Added dropwise to a mixture containing 100 g of commercial 2-thiopyrimidin-4-ol, 60 g of sodium hydroxide in 800 ml of water were 38 ml of methyl iodide. The reaction mixture was left stirring at ambient temperature for 24 hours. The solution was acidified with 135 ml of acetic acid and left for 24 hours in a refrigerator. The white precipitate was filtered and washed several times with cold water. After drying, 60 g of the target compound was obtained.

### Stage 2: 2-Anilinopyrimidin-4-ol

39 g of 2-(methylthio)pyrimidin-4-ol were dissolved in 500 ml of de DMF containing 30 ml of aniline. The reaction mixture was left stirring under reflux for 24 hours. After the usual treatment, 35.81 g of the target compound was obtained.

### Stage 3: 4-Chloro-N-phenylpyrimidin-2-amine

A solution containing 15 g of 2-anilinopyrimidin-4-ol in 75 ml of POCl₃ was brought to 110° C. for 2 hours. After evaporating the POCl₃, the crude reaction product was decanted into an iced solution of Na₂CO₃. 16.3 g of the target product were obtained by filtration of the precipitate.

### Stage 4: Hydrochloride of 2-[(2-chloropyrimidin-4-yl)-amino]benzenesulphonyl chloride

Added in small amounts, under a stream of nitrogen, to a three-necked, round-bottomed flask containing the chlorosulphonic acid at 0° C. were 16.2 g of 4-chloro-N-phenylpyrimidine-2-amine while maintaining the temperature around 0° C. The reaction mixture was left at ambient temperature for 18 h. The mixture was poured dropwise (carefully) over ice. The precipitate obtained was filtered and washed with distilled water. After dissolving the solid in 1 l of ethyl acetate, drying over Na₂SO₄ and concentrating under vacuum, a whitish oil was obtained. This oil precipitated after dispersion in 200 ml of ether. 7.6 g of hydrochloride of 2-[(2-chloropyrimidin-4-yl)amino]benzenesulphonyl chloride were obtained by filtering the ether suspension.

MH⁺=304.2.

### Procedure 1b: Preparation of the hydrochloride of 2-[(5-fluoro-2-chloropyrimidin-4-yl)amino]benzenesulphonyl

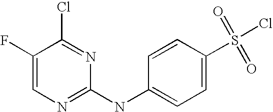

In order to prepare this compound, the same steps as those from procedure 1a were used by replacing, in stage 1 of this procedure 1a, 2-(methylthio)pyrimidin-4-ol with 2-chloro-5-fluoro-pyrimidin-4-ol.

### Procedure 2: Preparation of the Amines

### Procedure 2a: 4-Pyrrolidin-1-ylmethylpiperidin-4-ol

### Step 1: 1-Oxa-6-aza-spiro[2.5]octane-6-carboxylic acid tert-butyl ester

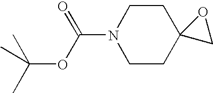

Added to a suspension of 15 g of 4-oxo-piperidine-1-carboxylic acid tert-butyl ester in 150 ml of toluene were 18.22 g of trimethylsulphoxonium iodide and 485 mg of tetrabutylammonium bromide. A solution of 4.5 g of sodium hydroxide and 20 ml of water was added dropwise. The mixture was left stirring for 3 h at 80° C. It was taken up with toluene, decanted, washed with water, dried and concentrated to dryness. After chromato-graphing on a silica column (DCM/AcOEt: 90/10), 13 g of the target product was obtained.

### Step 2: 4-Hydroxy-4-pyrrolidin-1-ylmethylpiperidine-1-carboxylic acid tert-butyl ester

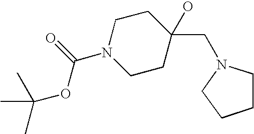

2.2 g of the product obtained in the preceding step were put into solution with 1.46 g of pyrrolidine and 25 ml of EtOH in a sealed tube. The reaction medium is heated at 75° C. for 18 h. After concentrating to dryness, taking up with water, extraction with DCM, drying and concentrating, 2.9 g of the desired product were obtained.

### Step 3: 4-Pyrrolidin-1-ylmethylpiperidin-4-ol dihydrochloride

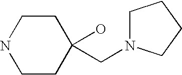

In a dioxane/MeOH mixture (50 ml), 2.9 g of the above product, in the presence of a 4 M HCl solution in the dioxane, were stirred for 4 h at RT. It was concentrated under vacuum and triturated in isopropyl ether and the solid was filtered which was used “as is” in the coupling reaction with sulphonyl chloride.

### Procedure 2b: 4-(2-(Rac)-methylpyrrolidin)-1-ylmethyl-piperidin-4-ol

According to the operating process described in stage 2 and 3 of procedure 2a, the target amine was prepared from the epoxide from step 1 of procedure 2a and from 2-(Rac)-methylpyrrolidine.

### Procedure 2c: 4-(2-R-methylpyrrolidin)-1-ylmethylpiperidin-4-ol

According to the operating process described in stage 2 and 3 of procedure 2a, the target amine was prepared from the epoxide from step 1 of procedure 2a and from 2-R-methylpyrrolidine.

### Procedure 2d: 4-(2-S-methylpyrrolidin)-1-ylmethylpiperidin-4-ol

According to the operating process described in stage 2 and 3 of procedure 2a, the target amine was prepared from the epoxide from step 1 of procedure 2a and from 2-S-methylpyrrolidine.

### Example 1

### 4-[4-(5-Fluoro-2,3-dihydroindol-1-yl)pyrimidin-2-ylamino]-N-piperidin-4-yl-N-(2-pyrrolidin-1-yl-ethyl)benzenesulphonamide

### Stage 1: 4-[[4-(4-Chloropyrimidin-2-ylamino)benzenesulphonyl]-(2-pyrrolidin-1-ylethyl)amino]piperidine-1-carboxylic acid tert-butyl ester

Added to a solution of 4 g of the chloride from procedure 1a in 300 ml of dichloromethane were 3.92 g of 4-(2-pyrrolidin-1-ylethylamino)piperidine-1-carboxylic acid tert-butyl ester), then 7 ml of diisopropylethylamine. The reaction mixture was left stirring at ambient temperature for 18 hours. The reaction mixture was concentrated to dryness and taken up with a 10% solution of K₂CO₃. After extracting with ethyl acetate, the organic phase was washed with a saturated solution of NaCl, then dried over Na₂SO₄. The crude reaction product was purified by chromatography on a silica column (CH₂Cl₂ then 10% of methanol in CH₂Cl₂); 6.67 g of the target compound were obtained.

### Stage 2: 4-[4-(5-Fluoro-2,3-dihydroindol-1-yl)pyrimidin-2-ylamino]-N-piperidin-4-yl-N-(2-pyrrolidin-1-yl-ethyl)benzenesulphonamide

According to the procedure described in stage 2 of procedure 1a, starting from 9.2 g of the compound obtained in stage 1 and from 2.68 g of 5-fluoro-2,3-dihydro-1H-indole, 4.67 g of the target product were obtained. (In the course of this reaction step, two successive reactions were carried out, the aromatic nucleophilic substitution reaction and also a decarboxylation reaction).

MH⁺=566.3

Melting point=269.5° C. (isopropyl ether/dichloromethane)

¹H NMR (DMSO): 1.15-1.55 (m, 4); 1.66 (m, 4); 2.4-2.8 (unresolved peak, 8); 3.14 (m, 4); 3.74 (m, 2); 3.85 (dl, 2); 4.04 (t, 2); 6.36 (t, 1); 6.93 (td, 1); 7.09 (dd, 1); 7.71 (d, 2); 7.95 (d, 2); 8.19 (d, 1); 8.47 (m, 1); 9.72 (sl, 1).

### Example 2

### N-(1-Cyclopropylpiperidin-4-yl)-4-[4-(5-fluoro-2,3-dihydroindol-1-yl)pyrimidin-2-ylamino]-N-(2-pyrrolidin-1-ylethyl)benzenesulphonamide

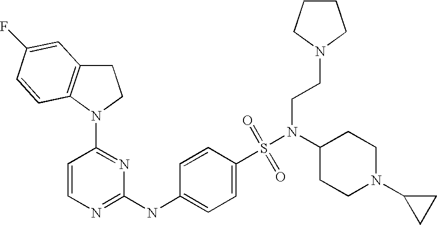

600 mg of the compound obtained in Example 1 were reacted with 555 mg of (1-ethoxycyclopropoxy)trimethylsilane in the presence of 200 mg of sodium cyanoborohydride in 10 ml of methanol. The reaction medium was left at 60° C. for 38 hours. At the end of the reaction, 10 ml of a 10% solution of Na₂CO₃ was added. A beige precipitate formed. After filtration, 469 mg of the target product were thus obtained.

MH⁺=606.2

Melting point=215.4° C. (isopropylether/dichloromethane)

¹H NMR (DMSO): 0.22 (m, 2); 0.36 (m, 5); 1.29-1.59 (unresolved peak, 5); 1.65 (m, 4); 2.13 (t, 2); 2.45 (m, 4); 2.57 (t, 2); 2.57 (t, 2); 2.89 (d, 2); 3.16 (t, 2); 3.23 (t, 2); 3.55 (t, 1); 4.09 (t, 2); 6.38 (d, 1); 7.13 (d, 1); 7.73 (d, 1); 7.97 (d, 2); 8.23 (d, 1); 8.50 (sl, 1); 9.72 (sl, 1).

### Example 3

### 4-[4-(5-Fluoro-2,3-dihydroindol-1-yl)-pyrimidin-2-ylamino]-N-(2-pyrrolidin-1-ylethyl)-N-[1-(4,4,4-trifluorobutyl)piperidin-4-yl]benzenesulphonamide

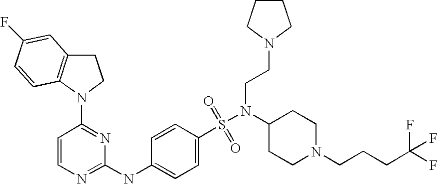

A reductive amination reaction was carried out starting from 600 mg of the compound obtained in Example 1 that was reacted with 161 mg of 4,4,4-trifluorobutyraldehyde. Thus, 300 mg of target product were obtained.

MH⁺=676.2

Melting point=198° C. (isopropyl ether/dichloromethane)

¹H NMR (DMSO): 1.38 (d, 2); 1.51 to 1.63 (unresolved peak, 4); 1.67 (m, 4); 1.89 (t, 2); 2.14 to 2.25 (unresolved peak, 2); 2.28 (t, 2); 2.47 (m, 4); 2.59 (t, 2); 2.80 (d, 2); 3.14 to 3.27 (unresolved peak, 4); 3.54 (m, 1); 4.09 (t, 2); 6.39 (d, 1); 6.94 (td, 1); 7.14 (d, 1); 7.73 (d, 2); 7.96 (d, 2); 8.24 (d, 1); 8.50 (dd, 1); 9.72 (sl, 1).

### Example 4

### 4-[4-(5-Fluoro-2,3-dihydroindol-1-yl)pyrimidin-2-ylamino]-N-(2-pyrrolidin-1-ylethyl)-N-(1-thiophen-3-ylmethylpiperidin-4-yl)benzenesulphonamide

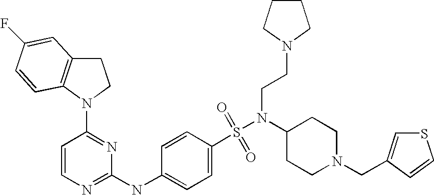

A reductive amination reaction was carried out starting from 600 mg of the compound obtained in Example 1 that was reacted with 110 ml of thiophene-3-carbaldehyde. Thus, 330 mg of target product were obtained.

MH⁺=662.1

Melting point=140° C. (isopropyl ether/dichloromethane)

¹H NMR (DMSO): 1.37 (d, 2); 1.58 (m, 2); 1.66 (m, 4); 1.90 (t, 2); 2.46 (m, 4); 2.58 (t, 2); 2.79 (d, 2); 3.10-3.27 (unresolved peak, 4); 3.42 (s, 2); 3.52 (m, 1); 4.08 (t, 2); 6.39 (d, 1); 6.94 (t, 1); 7.00 (d, 1); 7.14 (d, 1); 7.26 (s, 1); 7.45 (m, 1); 7.72 (d, 2); 7.96 (d, 2); 8.23 (d, 1); 8.51 (m, 1); 9.73 (sl, 1).

### Example 5

### 4-[4-(5-Fluoro-2,3-dihydroindol-1-yl)-pyrimidin-2-ylamino]-N-[1-(1-methyl-1H-pyrrol-2-ylmethyl)piperidin-4-yl]-N-(2-pyrrolidin-1-ylethyl)benzenesulphonamide

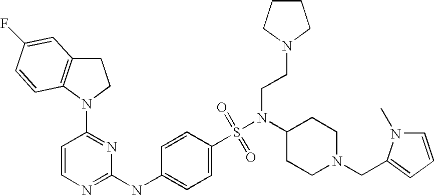

A reductive amination reaction was carried out starting from 400 mg of the compound obtained in Example 1 that was reacted with 92 mg of 1-methyl-1H-pyrrole-2-carbaldehyde. Thus, 50 mg of the target product were obtained.

MH⁺=659.2

Melting point=94° C. (isopropyl ether/dichloromethane)

¹H NMR (DMSO): 1.38 (d, 2); 1.54 (m, 2); 1.66 (sl, 2); 1.87 (t, 2); 2.45 (sl, 2); 2.57 (t, 2); 2.80 (d, 2); 3.18 (t, 2); 3.23 (t, 2); 3.29 (s, 2); 3.56 (sl, 2); 4.09 (t, 2); 5.83 (unresolved peak, 2); 6.39 (d, 2); 6.62 (s, 2); 6.95 (t, 2); 7.14 (d, 2); 7.73 (d, 2); 7.96 (d, 2); 8.24 (d, 2); 8.50 (m, 2); 9.73 (sl, 2).

### Example 6

### 4-[4-(5-Fluoro-2,3-dihydroindol-1-yl)-pyrimidin-2-ylamino]-N-(2-pyrrolidin-1-ylethyl)-N-[1-(3,3,3-trifluoropropyl)piperidin-4-yl]benzenesulphonamide

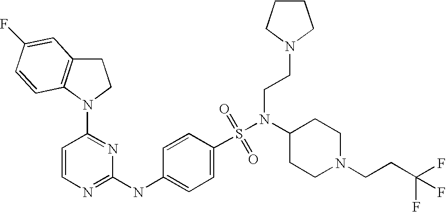

A reductive amination reaction was carried out starting from 1 g of the compound from Example 1 that was reacted with 238 mg of 3,3,3-trifluoropropaldehyde. Thus, 97 mg of target product were obtained.

MH⁺=662.1

Melting point=147° C. (isopropyl ether/dichloromethane)

¹H NMR (DMSO): 1.36 (d, 2); 1.57 (m, 2); 1.70 (sl, 4); 1.95 (t, 2); 2.27-2.79 (unresolved peak, 10); 2.85 (d, 2); 3.12-3.27 (unresolved peak, 4); 3.55 (m, 1); 4.09 (t, 2); 6.39 (d, 1); 6.95 (t, 1); 7.14 (d, 1); 7.74 (d, 2); 7.97 (d, 2); 8.24 (d, 1); 8.51 (m, 1); 9.74 (s, 1).

### Example 7

### 4-[4-(5-Fluoro-2,3-dihydroindol-1-yl)-pyrimidin-2-ylamino]-N-(2-pyrrolidin-1-ylethyl)-N-[1-(2,2,2-trifluoroethyl)piperidin-4-yl]benzenesulphonamide

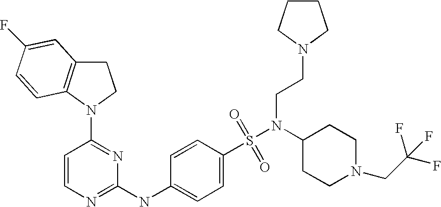

A nucleophilic substitution reaction was carried out starting from 1 g of the compound from Example 1, 0.4 g of 2,2,2-trifluoroethyltrifluoromethanesulphonamide and 0.3 g of NaHCO₃ in 50 ml of EtOH under reflux for 48 h. After the usual treatment and chromatography on an SiO₂ phase (DCM, 10% MeOH gradient), 153 mg of the target product were thus obtained.

MH⁺=648.2

Melting point=139.3° C. (isopropyl ether/dichloromethane)

¹H NMR (DMSO): 1.21-1.76 (unresolved peak, 4); 1.76-2.04 (unresolved peak, 4); 2.35 (t, 2); 2.89-3.46 (unresolved peak, 14); 3.6 (m, 1); 4.09 (t, 2); 6.4 (d, 1); 6.95 (t, 1); 7.15 (d, 1); 7.78 (d, 2); 8.01 (d, 2); 8.23 (d, 1); 8.52 (m, 1); 9.78 (s, 1)

### Example 8

### 4-[4-(5-Fluoro-2,3-dihydroindol-1-yl)pyrimidin-2-ylamino]-N-(2-pyrrolidin-1-ylethyl)-N-(tetrahydropyran-4-yl)benzenesulphonamide

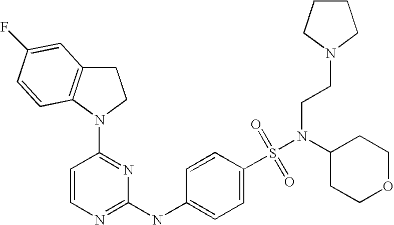

### Stage 1: 4-(4-Chloropyrimidin-2-ylamino)-N-(2-pyrrolidin-1-ylethyl)-N-(tetrahydropyran-4-yl)benzenesulphonamide

Following the operating method described in stage 1 of Example 1, starting from 3 g of the compound from procedure 1a and 1.956 g of the amine from Example 2 of procedure 2, 3 g of the target product were obtained.

### Stage 2: 4-[4-(5-Fluoro-2,3-dihydroindol-1-yl)-pyrimidin-2-ylamino]-N-(2-pyrrolidin-1-ylethyl)-N-(tetrahydropyran-4-yl)benzenesulphonamide

Following the operating method described in stage 2 of Example 1, starting from 1 g of the compound obtained in stage 1 and from 354 mg of 5-fluoro-2,3-dihydro-1H-indole, 138 mg of the target product were obtained.

MH⁺=567.3

Melting point=100° C. (isopropyl ether/dichloromethane)

¹H NMR (DMSO): 1.30 (d, 2); 1.52 (m, 2); 1.86 (m, 4); 1.92 (t, 2); 2.49 (m, 4); 2.61 (t, 2); 2.82 (d, 2); 3.15-3.28 (unresolved peak, 4); 3.49 (s, 2); 3.58 (m, 1); 4.10 (t, 2); 6.35 (d, 1); 6.84 (t, 1); 7.09 (d, 1); 7.29 (s, 1); 7.47 (m, 1); 7.72 (d, 2); 7.98 (d, 2); 8.29 (d, 1).

### Example 9

### 4-[4-(5-Fluoro-2,3-dihydroindol-1-yl)-pyrimidin-2-ylamino]-N-methyl-N-(1-methylpiperidin-4-yl)benzenesulphonamide

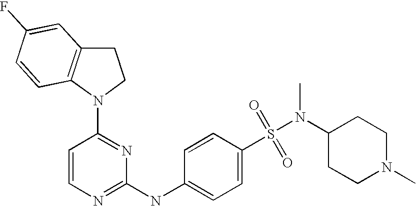

### Stage 1: 4-(4-Chloropyrimidin-2-ylamino)-N-methyl-N-(1-methylpiperidin-4-yl)benzenesulphonamide

According to the operating method described in stage 1 of Example 1, starting from 2 g of 4-[(4-chloropyrimidin-2-yl)amino]benzenesulphonyl chloride hydrochloride and from 0.96 ml of methyl (1-methylpiperidin-4-yl)amine, 1.88 g of the target product were obtained.

### Stage 2

Following the procedure described in stage 2 of Example 1, starting from 800 g of the compound obtained in stage 1 above and from 0.428 g of 5-fluoro-2,3-dihydro-1H-indole, 32 mg of the target product were obtained.

MH⁺=497.1

¹H NMR (DMSO): 1.21 (m, 2); 1.57 (m, 2); 1.86 (t, 2); 2.06 (s, 3); 2.65 (m, 5); 3.21 (m, 2); 3.59 (m, 1); 4.06 (t, 2); 6.37 (d, 1); 6.94 (t, 1); 7.13 (d, 1); 7.68 (d, 1); 7.98 (d, 2); 8.22 (d, 2); 8.50 (m, 1).

### Example 10

### 4-[4-(5-Chloro-2,3-dihydroindol-1-yl)-pyrimidin-2-ylamino]-N-(2-pyrrolidin-1-ylethyl)-N-(tetrahydropyran-4-yl)benzenesulphonamide

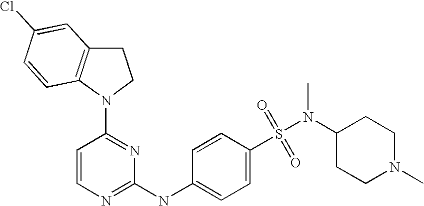

Following the procedure described in stage 2 of Example 1, starting from 800 g of the compound from stage 1 of Example 9 and from 0.428 g of 6-fluoro-2,3-dihydro-1H-indole, 105 mg of the target product were obtained.

MH⁺=514.3

¹H NMR (DMSO): 1.31 (m, 2); 1.59 (m, 2); 1.83 (t, 2); 2.04 (s, 3); 2.71 (m, 5); 3.18 (m, 2); 3.63 (m, 1); 4.06 (t, 2); 6.32 (d, 1); 6.98 (t, 1); 7.53 (d, 1); 7.68 (d, 1); 7.88 (d, 2); 8.39 (d, 2); 8.88 (m, 1).

### Examples 11 to 21

### Stage 1

The compound from stage 1 of Example 1 (100 mg) was dissolved in 3 ml of dimethylformamide and 115 mg of caesium carbonate, then 25.6 mg of 4-fluoroindole were added, all in a microwave tube. The reaction was brought to 110° C., using microwaves for 60 min. After cooling, the medium was directly purified by HPLC in an acid medium and, after freeze drying, 25.6 mg of the target product were obtained.

### Stage 2

The product obtained in stage 1 above was put in solution in methylene chloride at 20% of trifluoroacetic acid, 10 ml. After 6 h at ambient temperature, the medium was evaporated to dryness and the crude reaction product, dissolved in DMSO was purified by HPLC in an acid medium. After freeze-drying, 30 mg of product from Example 14 were obtained.

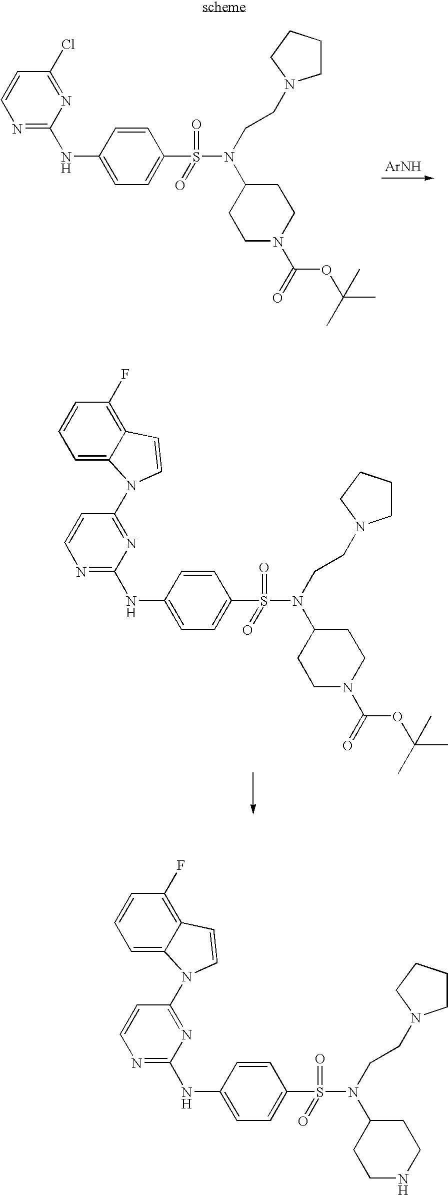

In the same manner, the products from Examples 11 to 13 and 15 to 21, described in the table below, were obtained either in the form of a formic acid salt or in a neutral form:

| Ex | STRUCTURE | MH⁺ | NAME |
|---|---|---|---|
| 11 | 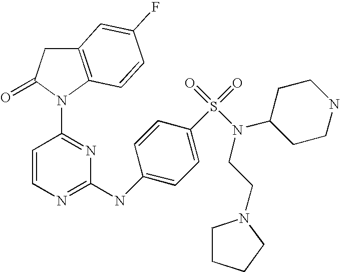 | 580.00 | 4-{[4-(6-fluoro-2- oxo-2,3-dihydro- 1H-indol-1-yl)- pyrimidin-2- yl]amino}-N- piperidin-4-yl-N- (2-pyrrolidin-1- ylethyl)- benzenesulphonamide formate (1:1) |
| 12 | 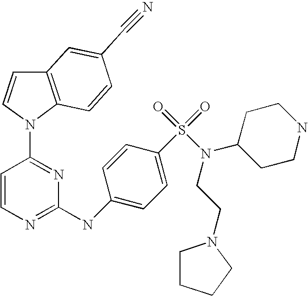 | 570.71 | 4-{[4-(5-cyano-1H- indol-1-yl) - pyrimidin-2- yl]amino}-N- piperidin-4-yl-N- (2-pyrrolidin-1- ylethyl)benzene- sulphonamide formate (1:1) |
| 13 | 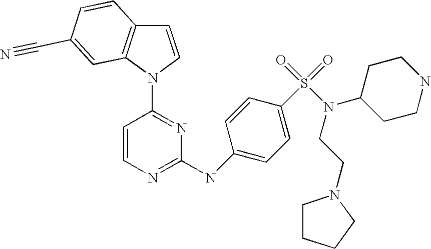 | 570.71 | 4-{[4-(6-cyano-1H- indol-1- yl)pyrimidin-2- yl]amino}-N- piperidin-4-yl-N- (2-pyrrolidin-1- ylethyl)benzene- sulphonamide formate (1:1) |
| 14 | 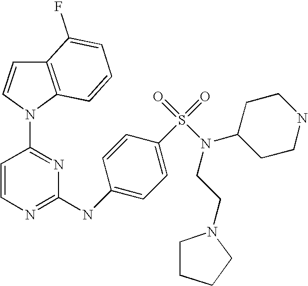 | 563.69 | 4-{[4-(4-fluoro- 1H-indol-1- yl) pyrimidin-2- yl]amino}-N- piperidin-4-yl-N- (2-pyrrolidin-1-ylethyl)benzene- sulphonamide formate (1:1) |
| 15 | 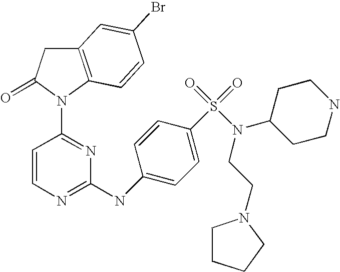 | 624.61 | 4-{[4-(5-bromo-1H- indol-1-yl) - pyrimidin-2- yl]amino}-N- piperidin-4-yl-N- (2-pyrrolidin-1- ylethyl)benzene sulphonamide formate (1:1) |
| 16 | 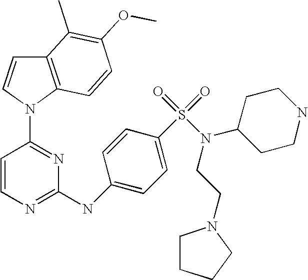 | 590.00 | 4-{[4-(5-methoxy- 4-methyl-1H-indol- l-yl)pyrimidin-2- yl]amino}-N- piperidin-4-yl-N- (2-pyrrolidin-1- ylethyl)benzene- sulphonamide |
| 17 | 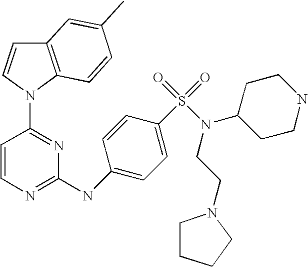 | 560.00 | 4{[4-(5-methyl- 1H-indol-1- yl)pyrimidin-2- yl]amino}-N- piperidin-4-yl-N- (2-pyrrolidin-1- ylethyl)benzene- sulphonamide |
| 18 | 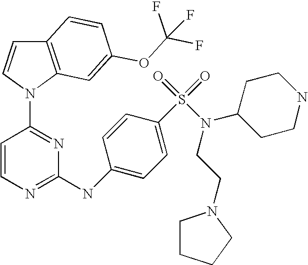 | 613.70 | N-piperidin-4-yl- N-(2-pyrrolidin-1- ylethyl)-4-({4-[6- (trifluoromethyl)- 1H-indol-1- yl]pyrimidin-2- yl}amino)- benzenesulphonamide formate (1:1) |
| 19 | 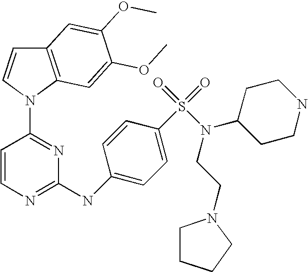 | 605.76 | 4-{[4-(5,6- dimethoxy-1H- indol-1- yl)pyrimidin-2- yl]amino}-N- piperidin-4-yl-N- (2-pyrrolidin-1- ylethyl)benzene- sulphonamide formate (1:1) |
| 20 | 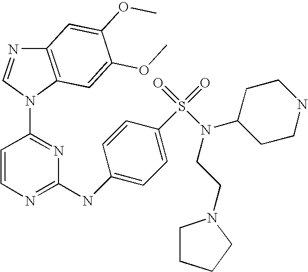 | 607.00 | 4-{[4-(5,6- dimethoxy-1H- benzimidazol-1- yl)pyrimidin-2- yl]amino}-N- piperidin-4-yl-N- (2-pyrrolidin-1- ylethyl)- benzenesulphonamide |
| 21 | 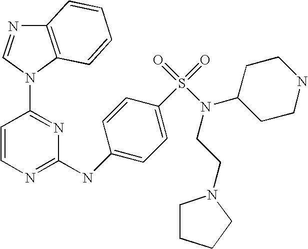 | 547.00 | 4-{[4-(1H- benzimidazol-1- yl)pyrimidin-2- yl]amino}-N- piperidin-4-yl-N- (2-pyrrolidin-1- ylethyl)benzene- sulphonamide |

Examples 22 to 33 were synthesized following the procedure described in stage 2 of Example 1 starting from the derivatives of 4-chloropyrimidine described below (procedure 3a, 3b, 3c, 3d) and the corresponding boronic acids.

### Procedure 3a: 1-[4-(4-Chloropyrimidin-2-ylamino)-benzenesulphonyl]-4-pyrrolidin-1-ylmethylpiperidin-4-ol

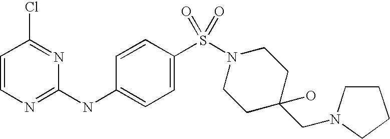

Following the procedure described in stage 1 of Example 1, starting from 3.8 g of the chloride from stage 4 of procedure 1a and from 3.2134 g of the amine obtained in procedure 2a, 5.15 g of the target product (91% purity) were obtained.

MH⁺=452.1

Similarly, for the procedures 3b, 3c, 3d below, the amines prepared in procedure 2b, 2c and 2d were used.

### Procedure 3b: 1-[4-(4-Chloropyrimidin-2-ylamino)-benzenesulphonyl]-4-(2-methylpyrrolidin)-1-ylmethylpiperidin-4-ol

### Procedure 3c: 1-[4-(4-Chloropyrimidin-2-ylamino)-benzenesulphonyl]-4-(2-R-methylpyrrolidin)-1-ylmethylpiperidin-4-ol

### Procedure 3d: 1-[4-(4-Chloropyrimidin-2-ylamino)-benzenesulphonyl]-4-(2-S-methylpyrrolidin)-1-ylmethylpiperidin-4-ol

| | | | | MP |
|---|---|---|---|---|
| Ex | Structure | Name | MH⁺ | (° C.) |
| 22 | 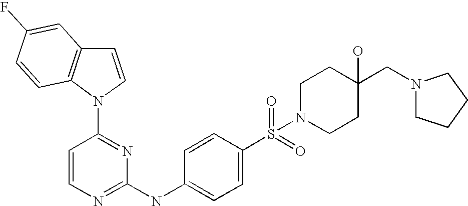 | 1-{4-[4-(5- Fluoroindol-1- yl)pyrimidin- 2-yl- amino]benzene sulphonyl}-4- pyrrolidin-1- ylmethyl- piperidin-4-ol | 452.1 | 209 |
| 23 | 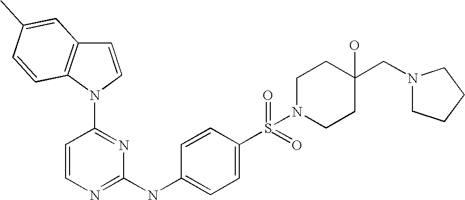 | 1-{4-[4- (5- Methylindol-1- yl)pyrimidin-2- ylamino]benzene- sulphonyl} - 4-pyrrolidin-1- ylmethyl- piperidin-4-ol | 547.2 | 102.6 |
| 24 | 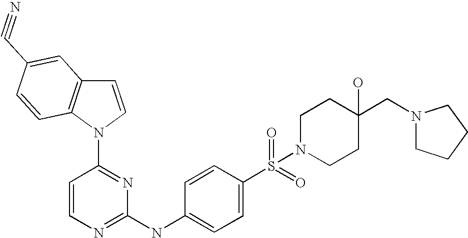 | 1-{2-[4-(4- Hydroxy-4- pyrrolidin-1-yl- methylpiperidine-1- sulphonyl)phenyl- amino]pyrimidin- 4-yl}-1H- indole-5- carbonitrile | 558.1 | 219.9 |
| 25 | 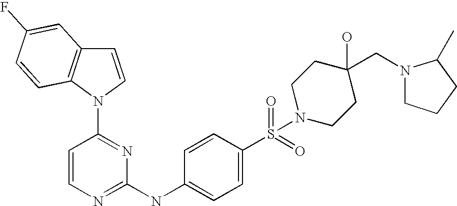 | 1-{4-[4-(5- Fluoroindol-1- yl)pyrimidin-2- ylamino]benzene- sulphonyl}- 4-(2-methyl- pyrrolidin-1- ylmethyl) piperidin-4-ol | 565.2 | 230 |
| 26 | 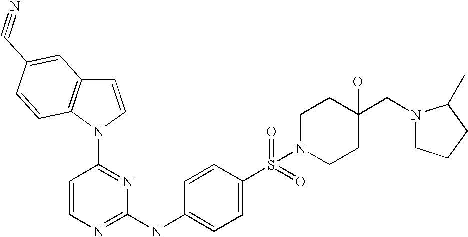 | 1-(2-{4-[4- Hydroxy-4-(2- methyl- pyrrolidin-1- ylmethyl) piperidine-1- sulphonyl] - phenylamino} pyrimidin-4- yl)-1H- indole-5- carbonitrile | 572 | 222 |
| 27 | 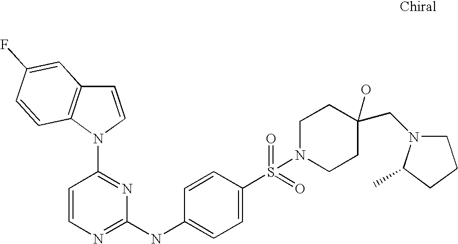 | 1-{4-[4-(5- Fluoroindol-1- yl)pyrimidin-2- ylamino]benzene- sulphonyl}- 4-((S)-2- methylpyrrolidin- 1-yl-methyl) piperidin-4-ol | 565.2 | 130 |
| 28 | 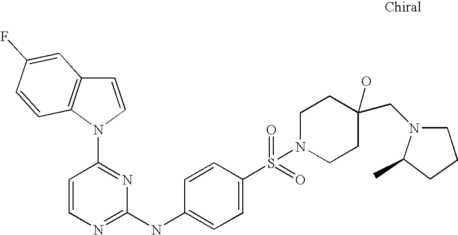 | 1-{4-[4-(5- Fluoroindol-1- yl)pyrimidin-2-yl- amino]benzene sulphonyl}-4- ((R)-2-methyl- pyrrolidin-1-yl- methyl) piperidin-4-ol | 565.1 | 216 |
| 29 | 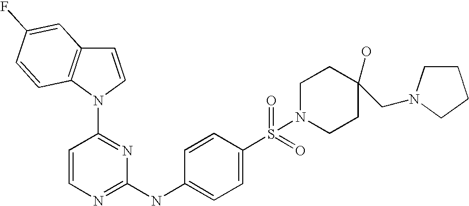 | 1-{4-[4-(4- Fluoroindol-1- yl)pyrimidin-2-yl- amino]benzene sulphonyl}-4- pyrrolidin-1- ylmethyl- piperidin-4-ol | 551.1 | 109.6 |
| 30 | 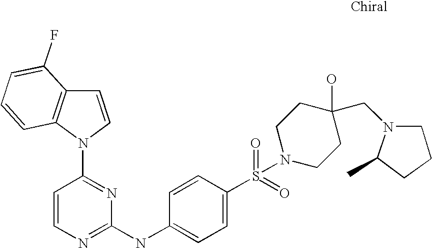 | 1-{4-[4-(4- Fluoroindol-1- yl)pyrimidin-2- ylamino]benzene- sulphonyl} - 4-((R)-2-methyl- pyrrolidin-1- ylmethyl) - piperidin-4-ol | 565.1 | 115.6 |
| 31 | 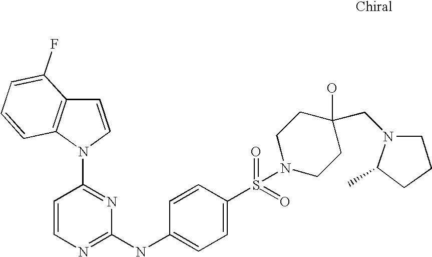 | 1-{4-[4-(4- Fluoroindol-1- yl)pyrimidin-2- ylamino]benzene- sulphonyl}- 4- ((S)-2-methyl- pyrrolidin-1- ylmethyl)- piperidin-4-ol | 565.1 | 114 |
| 32 | 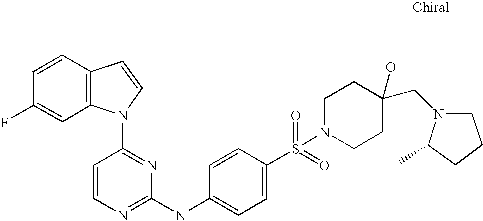 | 1-{4-[4-(6- Fluoroindol-1- yl)pyrimidin-2- ylamino]benzene- sulphonyl}- 4-((S)-2-methyl- pyrrolidin-1- ylmethyl) - piperidin-4-ol | 565 | 176; [α]_{D} = +30.6 |
| 33 | 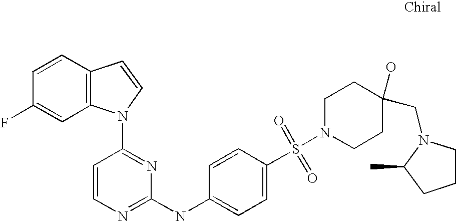 | 1-{4-[4-(6- Fluoroindol-1- yl)pyrimidin-2- ylamino]benzene- sulphonyl}- 4-((R)-2-methyl- pyrrolidin-1- ylmethyl)- piperidin-4-ol | 565 | 168; [α]_{D} = −25.6 |

### Example 34

### Pharmaceutical Composition

Tablets were prepared corresponding to the following formulation:

Product of Example 5 . . . 0.2 g

Excipient for a tablet made up to . . . 1 g

(breakdown of the excipient: lactose, talc, starch, magnesium stearate).

Example 5 is taken as example in the pharmaceutical preparation constituted by Example 22 above, it being possible for this pharmaceutical preparation to be produced differently as indicated above and if desired with other products in examples in the present patent application.

Pharmacological Part:

Protocols for Biochemical Trials on IKK

I) Evaluation of the Compounds with Regard to IKK1 and IKK2:

The compounds are tested for inhibition of IKK1 and IKK2 using a kinase test on a flash plate support. The test compounds are dissolved at 10 mM in DMSO and then diluted in kinase buffer (50 mM Tris, pH 7.4, containing 0.1 mM EGTA, 0.1 mM sodium orthovanadate and 0.1% p-mercaptoethanol).

Serial three-fold dilutions are carried out starting from this solution. 10 μl of each dilution are added to the wells of a 96-well plate in duplicate. 10 μl of kinase buffer are added to the control wells, which will serve for 0% inhibition, and 10 μl of 0.5 mM EDTA are added to the control wells (100% inhibition). 10 μl of the mixture IKK1 or IKK2 (0.1 μg/well), biotinylated 25-55 IKB substrate peptide and BSA (5 μg) are added to each well. To initiate the kinase reaction, 10 μl of the mixture of 10 mM magnesium acetate, 1 μM cold ATP and 0.1 μCi³³P-ATP are added to each well for a final volume of 30 μl. The reaction is then incubated at 30° C. for 90 min and then halted by the addition of 40 μl of 0.5 mM EDTA. After stirring, 50 μl are transferred to a flash plate covered with streptavidin.

30 min later, the wells are washed twice with a 50 mM Tris-EDTA, pH 7.5, solution and the radioactivity is determined on a MicroBeta counter.

The compounds of the invention tested in this trial show an IC₅₀ of less than 10 μM, which shows that they can be used for their therapeutic activity.

II) Evaluation of the Compounds with Regard to the Viability and the Proliferation of Tumor Cells:

The compounds according to the invention formed the subject of pharmacological trials which make it possible to determine their anticancer activity.

The compounds of formula (I) according to the present invention were tested in vitro on a sample group of tumor lines of human origin originating:
from breast cancer: MDA-MB231 (American Type Culture Collection, Rockville, Md., USA, ATCC-HTB26), MDA-A1 or MDA-ADR (referred to as multidrug resistant MDR line, and described by E. Collomb et al. in Cytometry, 12(1), 15-25, 1991), and MCF7 (ATCC-HTB22),
from prostate cancer: DU145 (ATCC-HTB81) and PC3 (ATCC-CRL1435),
from colon cancer: HCT116 (ATCC-CCL247) and HCT15 (ATCC-CCL225),
from lung cancer: H460 (described by Carmichael in Cancer Research, 47 (4), 936-942, 1987, and provided by the National Cancer Institute, Frederick Cancer Research and Development Center, Frederick, Md., USA),
from glioblastoma: SF268 (described by Westphal in Biochemical & Biophysical Research Communications, 132 (1), 284-289, 1985, and provided by the National Cancer Institute, Frederick Cancer Research and Development Center, Frederick, Md., USA),
from leukemia: CMLT1 (described by Kuriyama et al. in Blood, 74, 1989, 1381-1387, by Soda et al. in British Journal of Haematology, 59, 1985, 671-679, and by Drexler in Leukemia Research, 18: 1994, 919-927, and provided by DSMZ, Mascheroder Weg 1b, 38124, Braunschweig, Germany).

The cell proliferation and viability were determined in a test using 3-(4,5-dimethylthiazol-2-yl)-5-(3-carboxymethoxyphenyl)-2-(4-sulphophenyl)-2H-tetrazolium (MTS) according to Fujishita T. et al., Oncology, 2003, 64 (4), 399-406. In this test, the mitochondrial ability of the living cells to convert MTS to a colored compound is measured after incubating for 72 hours a compound of formula (I) according to the invention. The concentrations of compound according to the invention which result in a 50% loss of cell proliferation and viability (IC₅₀) are less than 10 μM, depending on the tumor line and the compound tested.

Thus, according to the present invention, it appears that the compounds of formula (I) bring about a loss of proliferation and viability of tumor cells with an IC₅₀ of less than 10 μM.

What is claimed is:

1) A compound of formula (I): 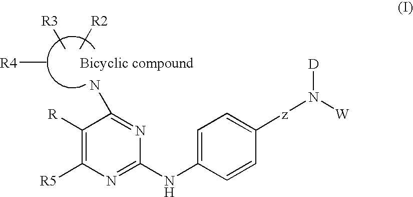
wherein:
bicyclic compound represents an unsaturated or partially unsaturated bicyclic radical composed of 9 or 10 ring members, containing one or two nitrogen atoms, bearing the radicals R2, R3 and R4 and, optionally, additionally bearing an oxo functional group;
R represents a hydrogen atom or a halogen atom;
R2, R3 and R4, which are identical or different, are chosen from hydrogen, halogen, CN, CONH₂, CONHalk and CON(alk)₂, and alkyl and alkoxy radicals that are optionally substituted by one or more halogen, CN, CONH₂, CONHalk, CON(alk)₂, OH or OCH₃, wherein one or two of R2, R3 and R4 represent a hydrogen atom, or wherein R2, R3 and R4 all represent methoxy;
R5 represents hydrogen or halogen;
Z represents CO or SO₂;
and the radical —N(D)(W) is such that:
a) W represents a -ring(Y) radical;
and D represents hydrogen, cycloalkyl, or an alkyl, alkenyl or alkynyl radical, all optionally substituted by one or more identical or different radicals chosen from halogen, OR8 and NR8R9, the alkyl radical represented by D in addition is optionally substituted by a saturated or unsaturated 5-membered heterocyclic radical attached via a carbon atom and optionally substituted by one or more radicals chosen from halogen, alkyl, and alkoxy;
and the ring(Y) is monocyclic or bicyclic, has from 4 to 10 ring members and is saturated or partially saturated, wherein Y represents an oxygen atom, a sulphur atom optionally oxidized by one or two oxygen atoms, NR10, C═O or its dioxolane as protective group for the carbonyl functional group, CF₂, CH—OR8 or CH—NR8R9;
wherein when Y represents NR10, the ring(Y) can comprise a carbon bridge composed of 1 to 3 carbons;
R10 represents hydrogen, cycloalkyl, or an alkyl, CH₂-alkenyl or CH₂-alkynyl radical, all optionally substituted by a naphthyl radical or by one or more identical or different radicals chosen from halogen, hydroxyl, alkoxy, aryl and heteroaryl radicals, the alkyl radical represented by R10 is in addition optionally substituted by a hydroxyl, NR8R9, CONR8R9, phosphonate, alkylthio optionally oxidized to give sulphone, or heterocycloalkyl radical, wherein the aryl, heteroaryl and heterocycloalkyl radicals are optionally substituted; or
b) W and D form, with the nitrogen atom to which they are bonded, a ring (N): 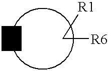
substituted on the same carbon atom by R1 and R6, wherein the ring (N) contains 4 to 7 ring members, is saturated and optionally comprises a carbon bridge composed of 1 to 3 carbons;
and wherein R1 and R6 represent one of the 6 following alternatives i) to vi):
i) R1 represents -X1-R7, wherein X1 represents —(CH₂)ₘ—; and
wherein R7 represents a heterocycloalkyl, aryl or heteroaryl ring, all optionally substituted;
and R6 represents hydrogen, hydroxyl, methyl, methoxy, —(CH₂)ₘOH, —CO—NRaRb, —CH₂—NraRb, —CO₂H or —CO₂alk;
ii) R1 represents -X2-R7, wherein X2 represents —O—, —O—(CH₂)ₘ—, —CH(OH)—(CH₂)ₙ—, —CO—, —CO—NRc-,
—CO—NRc-O—, —CH(NRaRb)—, —C═NOH—, —C═N—NH₂—, or —(CH2)ₙ₁-NRc-(CH₂)ₙ₂—; and R7 represents a heterocycloalkyl, aryl or heteroaryl ring, all optionally substituted;
and R6 represents hydrogen or a methyl radical;
iii) R1 represents —NRc-A, wherein A represents hydrogen or an alkyl radical containing 1 to 4 carbon atoms and is linear or branched starting from 3 carbon atoms and is optionally substituted by a radical chosen from —PO(OEt)₂, —OH, —Oalk, —CF₃, —CO—NR8R9 and SO₂-alk; and R6 represents hydrogen;
wherein when A represents a hydrogen atom, z represents CO;
iv) R1 represents —CH₂—NRc-A, wherein A represents a hydrogen atom or an alkyl radical containing 1 to 4 carbon atoms that is linear or branched starting from 3 carbon atoms and optionally substituted by a radical chosen from —PO(OEt)₂, —OH, —OEt, —CF₃, —CO—N(alk)₂ and SO₂-alk; and R6 represents hydrogen;
v) R1 represents —CO—N(Rc)-OR′c, and R6 represents hydrogen; or
vi) R1 represents X3-R7, wherein X3 represents —CH(OH)—(CH₂)n-, —CO—, —CH(NRaRb)—, —C═NOH—, or —C═N—NH₂—;
and R7 represents a heterocycloalkyl, aryl or heteroaryl ring, all optionally substituted;
and R6 represents hydrogen, hydroxyl, methyl, methoxy, —(CH₂)ₘOH, —CO—NRaRb, —CH₂—NraRb or —CO₂alk;
n, n1 and n2, which are identical or different, represent an integer from 0 to 3;
m represents an integer from 1 to 3;
Rc and R′c, which are identical or different, represent hydrogen, an alkyl radical containing from 1 to 4 carbon atoms optionally substituted by one or more halogen atoms;
R8 represents hydrogen, alkyl, cycloalkyl or heterocycloalkyl, which are optionally substituted by one or more radicals chosen from halogen, hydroxyl, alkoxy, NH₂, NHalkyl, N(alkyl)₂, —CONH₂, —CONHalkyl and
—CON(alkyl)₂, wherein the alkyl radicals represented by R8 are in addition optionally substituted by a phosphonate radical, an alkylthio radical optionally oxidized to sulphone, an aryl radical or a saturated or unsaturated, optionally substituted, heterocyclic radical;
NR8R9 is such that R8 and R9, which are identical or different, are chosen from the values of R8;
or R8 and R9 form, with the nitrogen atom to which they are bonded, an optionally substituted cyclic amine which can optionally include one or two other heteroatoms chosen from O, S, N and NRc; and wherein
all of the above aryl, naphthyl, phenyl, heterocyclic, heterocycloalkyl and heteroaryl radicals and also the cyclic amine which can be formed by R8 and R9 with the nitrogen atom to which they are bonded are optionally substituted by one or more identical or different radicals chosen from halogen; hydroxyl; cyano; NR8R9; and alkyl, cycloalkyl, alkoxy, phenyl, heterocycloalkyl and heteroaryl radicals, which are optionally substituted by one or more identical or different radicals chosen from halogen, hydroxyl, alkoxy, alkyl, hydroxyalkyl, alkoxyalkyl, CN, CF₃, OCF₃, and NRaRb;
NRaRb is such that Ra and Rb, which are identical or different, represent a hydrogen, an alkyl radical containing from 1 to 4 carbon atoms, or a cycloalkyl radical, wherein the alkyl and cycloalkyl radicals are optionally substituted by one or more identical or different radicals chosen from halogen, hydroxyl, alkoxy, NH₂, NHalkyl and N(alkyl)₂; or Ra and Rb form, with the nitrogen atom to which they are bonded, a cyclic amine which may optionally contain one or more other heteroatoms chosen from O, S, N and NRc, the cyclic amine is optionally substituted by one or more identical or different radicals chosen from halogen; oxo; hydroxyl; and alkyl optionally substituted by one or more halogen atoms; or by a methyl and a hydroxyl substituted on the same carbon; and wherein
all of the heterocyclic, heterocycloalkyl and heteroaryl radicals above are composed of 4 to 10 ring members, except where specified, and contain 1 to 4 heteroatoms chosen from O, optionally oxidized S, N and NRc;
or an acid addition salt thereof.

2) The compound of formula (I) according to claim 1, wherein R represents a halogen atom;
or an acid addition salt thereof.

3) The compound of formula (I) according to claim 1, wherein R represents a hydrogen atom;
or an acid addition salt thereof.

4) The compound of formula (I) according to claim 1, wherein:
R2, R3 and R4, which are identical or different, are chosen from hydrogen, halogen, CN, and alkyl and alkoxy radicals that are optionally substituted by one or more halogen, CN, CONH₂, CONHalk, or CON(alk)₂ radicals, wherein one or two of R2, R3 and R4 represent a hydrogen atom or R2, R3 and R4 all represent methoxy;
or an acid addition salt thereof.

5) The compound of formula (I) according to claim 1, wherein:
R2, R3 and R4, which are identical or different, represent hydrogen, halogen, CN or an alkyl or alkoxy radical optionally substituted by one or more halogen atoms;
and the radical —N(D)(W) is such that:
a) W represents a -ring(Y), wherein -ring(Y) and D are as defined in claim 1; or
b) W and D form, with the nitrogen atom to which they are bonded, a ring (N): 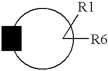
which is substituted on the same carbon atom by R1 and R6, contains 4 to 7 ring members, is saturated and optionally comprises a carbon bridge composed of 1 to 3 carbons,
wherein R1 and R6 represent one of the 5 following alternatives i) to v):
i) R1 represents -X1-R7, wherein X1 represents —(CH₂)ₘ—, and R7 represents a heterocycloalkyl, aryl or heteroaryl ring, all optionally substituted;
and R6 represents hydrogen, hydroxyl, —(CH₂)ₘOH, —CO—NRaRb, —CH₂—NraRb, or —CO₂alk;
ii) R1 represents -X2-R7, wherein X2 represents —O—, —O—(CH₂)ₘ—, —CH(OH)—(CH₂)ₙ—, —CO—, —CO—NRc-,
—CO—NRc-O—, —CH(NRaRb)—, —C═NOH—, —C═N—NH₂—, or —(CH2)ₙ₁-NRc-(CH₂)ₙ₂—; and R7 represents a heterocycloalkyl, aryl or heteroaryl ring, all optionally substituted;
and R6 represents hydrogen;
iii) R1 represents —NRc-A, wherein A represents a hydrogen atom or an alkyl radical containing 1 to 4 carbon atoms that is linear or branched starting from 3 carbon atoms and is optionally substituted by a radical chosen from —PO(OEt)₂, —OH, —Oalk, —CF₃, —CO—NR8R9 and SO₂-alk; and R6 represents hydrogen;
wherein when A represents a hydrogen, z represents CO;
iv) R1 represents —CH₂—NRc-A, wherein A represents a hydrogen, an alkyl radical containing 1 to 4 carbon atoms that is linear or branched starting from 3 carbon atoms and optionally substituted by a radical chosen from
—PO(OEt)₂, —OH, —OEt, —CF₃, —CO—N(alk)₂ and SO₂-alk; and R6 represents hydrogen; or
v) R1 represents —CO—N(Rc)-OR′c, and R6 represents hydrogen;
wherein n, n1, n2, m, Rc, R′c, R8 and R9 are as defined in claim 1;
NRaRb is such that either Ra and Rb, which are identical or different, represent hydrogen, an alkyl radical containing from 1 to 4 carbon atoms, or a cycloalkyl radical, wherein the alkyl and cycloalkyl radicals are optionally substituted by one or more identical or different radicals chosen from halogen, hydroxyl, alkoxy, NH₂, NHalkyl and N(alkyl)₂; or Ra and Rb form, with the nitrogen atom to which they are bonded, a cyclic amine which may optionally contain one or more other heteroatoms chosen from O, S, N and NRc, wherein the cyclic amine is optionally substituted by one or more identical or different radicals chosen from halogen and alkyl radicals optionally substituted by one or more halogen atoms;
or an acid addition salt thereof.

6) The compound of formula (I) according to claim 1 having the formula (IA): 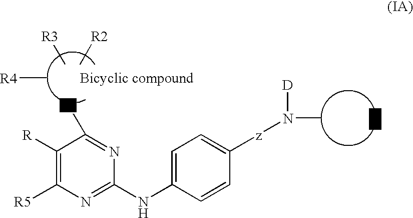
wherein bicyclic compound, R, R2, R3, R4, R5, z, D and ring (Y) are as defined in claim 1,
or an acid addition salt thereof.

7) The compound of formula (IA) according to claim 6, wherein:
D represents hydrogen or a linear or branched alkyl radical containing from 1 to 4 carbon atoms and optionally substituted by NH₂; and
Y represents NR10, wherein R10 represents a linear or branched alkyl radical containing 1 to 6 carbon atoms optionally substituted by a radical chosen from halogen, hydroxyl, phosphonate, sulphone, phenyl and saturated or unsaturated heterocyclic, monocyclic or bicyclic radicals, wherein the phenyl and heterocyclic radicals are optionally substituted;
or an acid addition salt thereof.

8) The compound of formula (IA) according to claim 6, wherein D represents CH₃;
or an acid addition salt thereof.

9) The compound of formula (IA) according to claim 6, wherein:
D represents a linear or branched alkyl radical containing from 1 to 4 carbon atoms optionally substituted by NH₂; and
Y represents NR8R9, wherein R8 represents a hydrogen atom or an alkyl radical, and R9 represents a linear or branched alkyl radical containing 1 to 6 carbon atoms and is optionally substituted by a radical chosen from halogen, hydroxyl, phosphonate, sulphone, phenyl and saturated or unsaturated heterocyclic, monocyclic or bicyclic radicals, wherein the phenyl and heterocyclic radicals are optionally substituted,
or an acid addition salt thereof.

10) The compound of formula (I) according to claim 1 having the formula (IB): 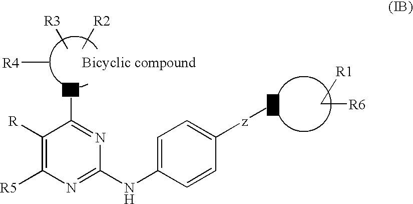
wherein bicyclic compound, R, R1, R2, R3, R4, R5, z, and ring (N) are as defined in claim 1,
or an acid addition salt thereof.

11) The compound of formula (IB) according to claim 10, wherein:
R2, R3 and R4, which are identical or different, represent a hydrogen atom, a halogen atom, CN or an alkyl or alkoxy radical optionally substituted by one or more halogen atoms;
or an acid addition salt thereof.

12) The compound of formula (IB) according to claim 10, wherein:
R1 represents -X1-R7, wherein X1 represents —(CH₂)ₘ—, and R7 represents a heterocycloalkyl, aryl or heteroaryl ring, all optionally substituted;
and R6 represents a hydrogen, hydroxyl, —(CH₂)ₘOH, —CO—NRaRb, —CH₂—NRaRb or —CO₂alk radical;
wherein m, n and NRaRb are as defined in claim 10;
or an acid addition salt thereof.

13) The compound of formula (IB) according to claim 10, wherein:
R1 represents -X2-R7, wherein X2 represents —O—, —O—(CH₂)ₘ—, —CH(OH)—(CH₂)ₙ—, —CO—, —CO—NRc-, —CO—NRc-O—, —CH(NRaRb)—, —C═NOH—, —C═N—NH₂—, or —(CH2)ₙ₁-NRc-(CH₂)ₙ₂—; and R7 represents a heterocycloalkyl, aryl or heteroaryl ring, all optionally substituted;
and R6 represents hydrogen;
or an acid addition salt thereof.

14) The compound of formula (IB) according to claim 10, wherein:
R1 represents —NRc-A, wherein A represents a hydrogen atom or an alkyl radical containing 1 to 4 carbon atoms that is linear or branched starting from 3 carbon atoms and optionally substituted by a radical chosen from —PO(OEt)₂, —OH, —Oalk, —CF₃, —CO—NR8R9 and SO₂-alk, and R6 represents hydrogen, and wherein when A represents a hydrogen atom, z represents CO;
or R1 represents —CH₂—NRc-A, wherein A represents a hydrogen atom or an alkyl radical containing 1 to 4 carbon atoms that is linear or branched starting from 3 carbon atoms and optionally substituted by a radical chosen from —PO(OEt)₂, —OH, —OEt, —CF₃, —CO—N(alk)₂ and SO₂-alk, and R6 represents hydrogen;
or R1 represents —CO—N(Rc)-OR′c, and R6 represents hydrogen;
wherein Rc, R′c and NR8R9 are as defined in claim 10;
or an acid addition salt thereof.

15) The compound of formula (IA) according to claim 6, wherein:
bicyclic compound represents an unsaturated or partially unsaturated bicyclic radical composed of 9 ring members, containing one or two nitrogen atoms, bearing the radicals R2, R3 and R4 and, optionally, additionally bearing an oxo functional group;
R2, R3 and R4, which are identical or different, represent hydrogen, halogen, CN or an alkyl or alkoxy radical that are optionally substituted by one or more halogen atoms;
D represents a hydrogen, a cycloalkyl radical, or an alkyl radical optionally substituted by one or more identical or different radicals chosen from halogen, OR8 and NR8R9;
the ring(Y) is monocyclic or bicyclic, has from 4 to 10 ring members and is saturated or partially saturated, wherein Y represents an oxygen atom, a sulphur atom optionally oxidized by one or two oxygen atoms, NR10, C═O, CF₂, CH—OR8 or CH—NR8R9;
R10 represents hydrogen or alkyl optionally substituted by one or more identical or different radicals chosen from halogen, hydroxyl, alkoxy, phenyl and heteroaryl, wherein the phenyl and heteroaryl radicals are optionally substituted by one or more identical or different radicals chosen from halogen, hydroxyl, alkoxy, alkyl, hydroxyalkyl, alkoxyalkyl, CF₃, NH₂, NHalk and N(alk)₂;
the heteroaryl radicals are composed of 5 to 7 ring members and include 1 to 3 heteroatoms chosen from O, S, N and NRc;
R8 represents hydrogen, a linear or branched alkyl radical having at most 4 carbon atoms or a cycloalkyl radical having from 3 to 6 ring members, wherein the alkyl and cycloalkyl radicals are optionally substituted by one or more identical or different radicals chosen from halogen, hydroxyl, NH2, NHalk and N(alk)₂;
NR8R9 is such that R8 and R9, which are identical or different, are chosen from the values of R8; or R8 and R9 form, with the nitrogen atom to which they are bonded, a cyclic amine chosen from pyrrolyl, piperidyl, morpholinyl, pyrrolidinyl, azetidinyl and piperazinyl radicals, wherein the piperazinyl radical is optionally substituted on the second nitrogen atom by an alkyl radical which is optionally substituted by one or more identical or different radicals chosen from halogen and hydroxyl;
or an acid addition salt thereof.

16) The compound of formula (IA) according to claim 6, wherein:
bicyclic compound represents an indolinyl, indolinone, indolyl or benzimidazole radical bearing the R2, R3 and R4 radicals,
R2, R3 and R4, which are identical or different, represent hydrogen, halogen, CN or a methyl or methoxy radical optionally substituted by one or more fluorine atoms;
R5 represents hydrogen, fluorine or chlorine;
Z represents SO₂ or CO;
D represents hydrogen or a cyclopropyl, methyl, ethyl, propyl or butyl radical which are optionally substituted by one or more identical or different radicals chosen from fluorine, hydroxyl, amino, alkylamino, dialkylamino, piperidinyl, morpholinyl, azetidinyl, piperazinyl, pyrrolidinyl and pyrrolyl;
the ring (Y) is chosen from cyclohexyl optionally substituted by amino; tetrahydropyranyl; dioxidothienyl; and pyrrolidinyl, piperidinyl and azepinyl radicals which are optionally substituted on their nitrogen atoms by a radical chosen from methyl, propyl, butyl, isopropyl, isobutyl, isopentyl and ethyl radicals, which radicals are optionally substituted by one or more radicals chosen from halogen, hydroxyl, phenyl, quinolyl, pyridyl optionally oxidized on its nitrogen atom, thienyl, thiazolyl, thiadiazolyl, tetrazolyl, pyrazinyl, furyl and imidazolyl, wherein the latter cyclic radicals are optionally substituted by one or more identical or different radicals chosen from halogen, hydroxyl, methyl and methoxy radicals;
or an acid addition salt thereof.

17) The compound of formula (IA) according to claim 6, wherein:
bicyclic compound represents an idolinyl, 2-oxoindolinyl, indolyl or benzimidazolyl radical bearing the R2, R3 and R4 radicals,
R2, R3 and R4, which are identical or different, represent hydrogen, fluorine, CF₃, CN, methyl or methoxy;
R5 represents hydrogen;
D represents a methyl radical or an ethyl radical which are optionally substituted by an amino, alkylamino, dialkylamino or pyrrolidinyl radical;
and the ring(Y) represents a cyclohexyl optionally substituted by amino, or a piperidinyl radical optionally substituted on its nitrogen atom by a methyl, propyl, butyl, isopropyl, isobutyl, isopentyl or ethyl radical, which radicals are optionally substituted by one or more halogen, or a radical chosen from hydroxyl; thiadiazolyl; tetrazolyl; phenyl optionally substituted by halogen; quinolyl; pyridy optionally oxidized on its nitrogen atom; furyl; and imidazolyl optionally substituted by alkyl;
or an acid addition salt thereof.

18) The compound of formula (IA) according to claim 6, wherein:
bicyclic compound represents an idolinyl, 2-oxoindolinyl, indolyl or benzimidazolyl radical bearing the R2, R3 and R4 radicals,
R2, R3 and R4, which are identical or different, represent hydrogen, fluorine, CF₃, CN, methyl or methoxy;
R5 represents hydrogen;
D represents a hydrogen, or a methyl radical or an ethyl radical optionally substituted by pyrrolidinyl;
the ring (Y) is chosen from tetrahydropyranyl; dioxidothienyl; and pyrrolidinyl, piperidinyl and azepinyl radicals optionally substituted on their nitrogen atoms, in the 2 or 3 position of the ring, by a methyl, ethyl, propyl or butyl radical, which radicals are optionally substituted by one or more halogen atoms or a phenyl, pyridyl, thienyl, thiazolyl, thiadiazolyl, pyrazinyl, furyl or imidazolyl radical;
or an acid addition salt thereof.

19) The compound of formula (IB) according to claim 10, wherein:
the ring(N) is selected from:
an azetidinyl or pyrrolidinyl ring substituted at position 3 by R1 and R6;
a piperidinyl or azepinyl ring substituted at position 3 or 4 by R1 and R6; and
a 8-azabicyclo[3.2.1]octan-3-yl, 6-azabicyclo[3.2.1]octan-3-yl or 3-azabicyclo[3.2.1]octan-8-yl) ring;
wherein R1 and R6 are as defined in claim 10;
or an acid addition salt thereof.

20) The compound of formula (IB) according to claim 10, wherein:
the ring(N) represents a pyrrolidinyl ring substituted at position 3 by R1 and R6, or ring(N) represents a piperidinyl ring substituted at position 3 or 4 by R1 and R6;
wherein R1 and R6 are as defined in claim 10;
or an acid addition salt thereof.

21) The compound formula (IB) according to claim 10, wherein:
bicyclic compound represents an indolinyl, 2-oxoindolinyl, indolyl or benzimidazole radical bearing the R2, R3 and R4 radicals,
R2, R3 and R4, which are identical or different, represent hydrogen, halogen, CN or a alkyl or alkoxy radical optionally substituted by one or more halogen;
the ring(N): 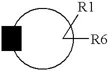
represents a pyrrolidinyl radical substituted at position 3 by R1 and R6, or a piperidinyl ring substituted at position 3 or 4 by R1 and R6,
wherein R1 and R6 represent one of the 5 following alternatives i) to v):
i) R1 represents -X1-R7, wherein X1 represents —CH₂, and R7 represents a heterocycloalkyl, phenyl or heteroaryl ring, all optionally substituted;
and R6 represents hydrogen, hydroxyl, —CH₂OH, —CO—NRaRb, or —CO₂Et;
ii) R1 represents -X2-R7, wherein X2 represents —O—, —CH(OH)—, —CH(OH)—CH₂—, —CO—, —CH(NRaRb)—, —C═NOH—, —C═N—NH₂—, or —(CH2)ₙ₁-NRc-(CH₂)ₙ₂—;
and wherein R7 represents a heterocycloalkyl, phenyl or heteroaryl ring, all optionally substituted;
and R6 represents hydrogen;
iii) R1 represents —NRc-A, wherein A represents hydrogen or an alkyl radical containing 1 to 4 carbon atoms that is linear or branched and optionally substituted by a radical chosen from —PO(OEt)₂, —OH, —OEt, —CF₃, —CO—NR8R9 and SO₂-alk; and R6 represents hydrogen;
wherein when A represents a hydrogen atom, z represents CO;
iv) R1 represents —CH₂—NRc-A, wherein A represents a hydrogen atom or an alkyl radical containing 1 to 4 carbon atoms that is linear or branched starting from 3 carbon atoms and optionally substituted by
SO₂-alk; and R6 represents hydrogen; or
v) R1 represents —CO—N(Rc)-OR′c, and R6 represents hydrogen;
wherein n, n1 and n2, which are identical or different, represent an integer from 0 to 2;
Rc and R′c, which are identical or different, represent a hydrogen atom or an alkyl radical containing 1 to 2 carbon atoms;
NRaRb is such that either Ra and Rb, being identical or different, represent hydrogen or an alkyl radical containing from 1 to 4 carbon atoms optionally substituted by one or more identical or different radicals chosen from halogen, hydroxyl, alkoxy, NH₂, NHalkyl and N(alkyl)₂;
or Ra and Rb form, with the nitrogen atom to which they are bonded, a morpholinyl or pyrrolidinyl radical optionally substituted by one or more identical or different radicals chosen from halogen and alkyl optionally substituted by one or more halogen atoms; and wherein
all of the heterocycloalkyl, phenyl and heteroaryl radicals are optionally substituted by one or more radicals, identical or different, chosen from halogen; hydroxyl; cyano; NR8R9; and alkyl, cycloalkyl, alkoxy, phenyl, heterocycloalkyl and heteroaryl radicals which are optionally substituted by one or more identical or different radicals chosen from halogen, hydroxyl, alkoxy, OCF₃, CH₃, —CH₂OH, CN, CF₃, OCF₃ and NRaRb; and
NR8R9 is such that either R8 and R9, which are identical or different, are such that R8 represents a hydrogen atom, a linear or branched alkyl radical containing at most 4 carbon atoms, or a cycloalkyl radical containing from 3 to 6 ring members, wherein the alkyl and cycloalkyl radicals are optionally substituted by one or more halogen or a hydroxyl radical; and R9 represents a hydrogen or an alkyl radical optionally substituted by one or more identical or different radicals chosen from halogen, hydroxyl, alkoxy, NH₂, NHalkyl, N(alkyl)₂, phenyl, heterocycloalkyl and heteroaryl radicals which are optionally substituted by one or more radicals chosen from halogen, hydroxyl, OCH₃, CH₃, —CH₂OH, CN, CF₃, OCF₃, NH₂, NHalk and N(alk)₂; or R8 and R9 form, with the nitrogen atom to which they are bonded, a cyclic amine chosen from pyrrolyl, piperidyl, morpholinyl, pyrrolidinyl, azetidinyl and piperazinyl optionally substituted by one or more alkyl radicals which are optionally substituted by one or more halogen;
or an acid addition salt thereof.

22) The compound of formula (IB) according to claim 10, wherein:
R1 represents -X-R7, wherein X1 represents —CH₂—, and R6 represents hydrogen, hydroxyl, CH₂—OH, —CO—N(CH₃)₂, —CO—NHCH₃, —CO—NH—(CH₂)₂—N(CH₃)₂, —CO₂Et, or CO₂H;
or R1 represents -X2-R7, wherein X represents —O—, —CHOH—, —CH(OH)—CH₂—, —CO—, —CHNH₂—, —NH—CH₂—, —N(CH₃)—CH₂— or CH₂—NH—CH₂—, and R6 represents hydrogen;
and wherein R7 is chosen from pyrrolidinyl, piperidinyl, piperazinyl, pyrimidinyl, morpholinyl, thiomorpholinyl, tetrahydrofuran, hexahydrofuranyl, phenyl, pyridyl, thienyl, thiazolyl, dithiazolyl, pyrazolyl, pyrazinyl, furyl, imidazolyl, pyrrolyl, oxazolyl, isoxazolyl, benzofuranyl, benzodihydrofuranyl, benzoxadiazolyl, benzothiadiazolyl, benzothienyl, quinolyl, and isoquinolyl radicals; and wherein
all of the radicals represented by R7 are optionally substituted by one or more identical or different radicals chosen from halogen, hydroxyl, methyl, methoxy, hydroxymethyl, alkoxymethyl, cyano, NH₂, NHalk, N(alk)₂, —CH₂—NH₂, —CH₂—NHalk, —CH₂—N(alk)₂, phenyl, morpholinyl and CH₂-morpholinyl radicals which are optionally substituted by one or more identical or different radicals chosen from halogen, hydroxyl, CH₃, OCH₃, —CH₂OH, CN, CF₃, OCF₃, NH₂, NHalk and N(alk)₂;
or an acid addition salt thereof.

23) The compound of formula (IB) according to claim 10, wherein:
R1 represents -X1-R7, wherein X1 represents —CH₂—, and R6 represents hydrogen, hydroxyl, CH₂—OH, —CO—N(CH₃)₂, —CO—NHCH₃, —CO—NH—(CH₂)₂—N(CH₃)₂ or —CO₂Et;
or R1 represents -X2-R7, wherein X2 represents —O—, —CHOH—, —CH(OH)—CH₂—, —CO—, —CHNH₂—, —NH—CH₂—, —N(CH₃)—CH₂— or CH₂—NH—CH₂—, and R6 represents hydrogen;
and wherein R7 is chosen from pyrrolidinyl, piperidinyl, piperazinyl, pyrimidinyl, morpholinyl, thiomorpholinyl, tetrahydrofuranyl, phenyl, pyridyl, thienyl, thiazolyl, dithiazolyl, pyrazolyl, pyrazinyl, furyl, imidazolyl, pyrrolyl, oxazolyl, isoxazolyl, benzodihydrofuranyl, benzoxadiazolyl, benzothiadiazolyl, benzothienyl, quinolyl, and isoquinolyl radicals; and wherein
all of the radicals represented by R7 are optionally substituted by one or more identical or different radicals chosen from halogen, hydroxyl, methyl, methoxy, hydroxymethyl, alkoxymethyl, cyano, NH₂, NHalk, N(alk)₂, —CH₂—NH₂, —CH₂—NHalk, —CH₂—N(alk)₂, phenyl, morpholinyl and CH₂-morpholinyl radicals which are optionally substituted by one or more identical or different radicals chosen from halogen, hydroxyl, CH₃, OCH₃, —CH₂OH, CN, CF₃, OCF₃, NH₂, NHalk and N(alk)₂ radicals;
or an acid addition salt thereof.

24) The compound of formula (I) according to claim 1, wherein:
R2, R3 and R4, which are identical or different, represent a hydrogen, halogen, CN, or a methyl or methoxy radical optionally substituted by one or more fluorine;
and R5 represents a hydrogen atom;
or an acid addition salt thereof.

25) The compound of formula (I) according to claim 1, wherein:
R2, R3 and R4, which are identical or different, are such that one represents a fluorine, and the other two independently represent hydrogen, fluorine or methyl; and
R5 represents a hydrogen atom;
or an acid addition salt thereof.

26) The compound of formula (I) according to claim 1, wherein z represents SO₂;
or an acid addition salt thereof.

27) The compound of formula (I) according to claim 1, wherein z represents CO;
or an acid addition salt thereof.

28) The compound formula (I) according to claim 1, selected from the group consisting of:
4-[4-(5-fluoro-2,3-dihydroindol-1-yl)pyrimidin-2-ylamino]-N-[1-(1-methyl-1H-pyrrol-2-ylmethyl)piperidin-4-yl]-N-(2-pyrrolidin-1-ylethyl)benzenesulphonamide;
4-[4-(5-fluoro-2,3-dihydroindol-1-yl)pyrimidin-2-ylamino]-N-(2-pyrrolidin-1-ylethyl)-N-(tetrahydropyran-4-yl)benzenesulphonamide;
4-[4-(5-fluoro-2,3-dihydroindol-1-yl)pyrimidin-2-ylamino]-N-methyl-N-(1-methylpiperidin-4-yl)benzenesulphonamide;
4-{[4-(5-cyano-1H-indol-1-yl)pyrimidin-2-yl]amino}-N-piperidin-4-yl-N-(2-pyrrolidin-1-ylethyl)benzenesulphonamide; and
4-{[4-(1H-benzimidazol-1-yl)pyrimidin-2-yl]amino}-N-piperidin-4-yl-N-(2-pyrrolidin-1-ylethyl)benzenesulphonamide;
or an acid addition salt thereof.

29) A method of preparing the compound of formula (I) as defined in claim 1, said method comprising converting a compound of formula (II) wherein R is as defined in claim 1 and R₅′ is as defined in claim 1 for R5 wherein the optional reactive functional groups are optionally protected: 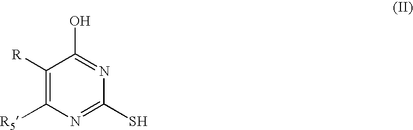
to a compound of formula (III): 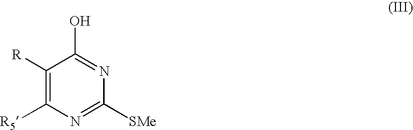
wherein R and R₅′ are as defined above;
following route a) for compounds of formula (I) wherein z=SO₂ or route b) for compounds of formula (I) wherein z=CO;
route a):
reacting the compound of formula (III) with the aniline of formula (IV): 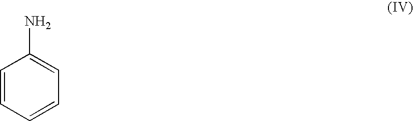
to obtain a compound of formula (V), wherein R and R₅′ are as defined above: 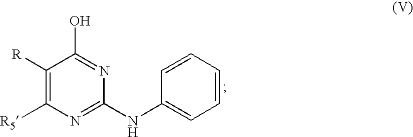
converting the compound of formula (V) to a compound of formula (VI): 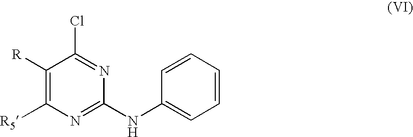
wherein R and R₅′ are as defined above,
reacting the compound of formula (VI) with chlorosulphonic acid SO₂(OH)Cl to obtain the corresponding compound of formula (VII): 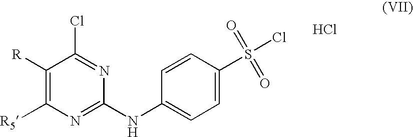
wherein R and R₅′ are as defined above;
reacting the compound of formula (VII) either with an amine of formula (VIII)1, wherein Y is defined in claim 1, and D′ is as defined in claim 1 for D wherein the optional reactive functional groups are optionally protected by protective groups: 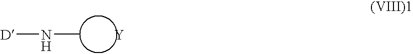
to obtain a compound of formula (IX) A1: 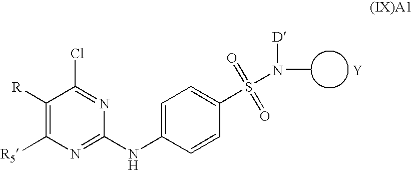
wherein R, R₅′, D′ and Y are as defined above;
or reacting the compound of formula (VII) with an amine of formula (VIII)2: 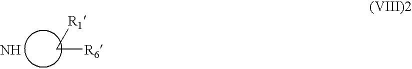
wherein R₁′ and R₆′ are as defined in claim 1 for R1 and R6, respectively, in which the optional reactive functional groups are optionally protected by protective groups,
to obtain a compound of formula (IX)A2: 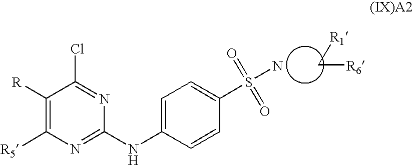
wherein R, R₁′, R₅′ and R₆′ are as defined above;
reacting the compound of formula (IX)A1 or the compound of formula (IX)A2 with a nitroheterocycle of formula (X) wherein R2, R3 and R4 are as defined in claim 1: 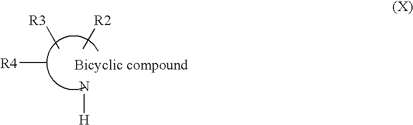
to respectively obtain a compound of formula (IA)1 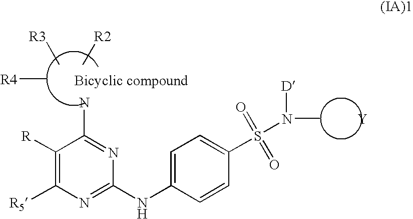
wherein R, R2, R3, R4, R₅′, D′ and Y are as defined above,
or a compound of formula (IA)2: 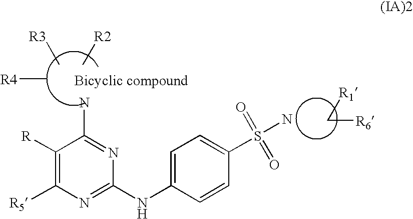
wherein R, R₁′, R2, R3, R4, R₅′ and R₆′ are as defined above,
route b)
reacting the compound of formula (III) as defined above with the methyl ester of 4-aminobenzoic acid to obtain the compound of formula (XI): 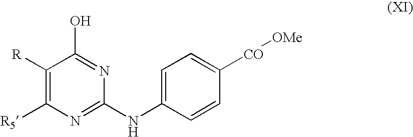
wherein R₅′ is as defined above;
reacting the compound of formula (XI) with a nitroheterocycle of formula (X) as defined above to obtain a compound of formula (XII): 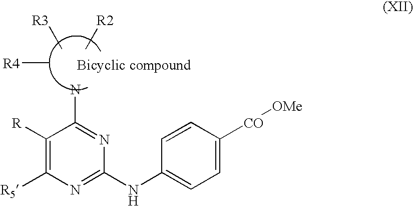
wherein R2, R3, R4 and R₅′ are as defined above;
converting the compound of formula (XII) to its corresponding acid of formula (XIII): 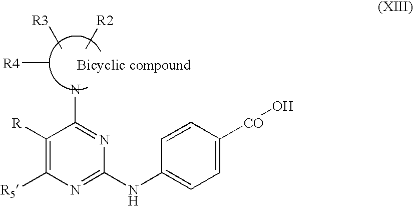
wherein R2, R3, R4 and R₅′ are as above;
reacting the compound of formula (XIII) with either an amine of formula (VIII)₁ as defined above to obtain a compound of formula (IB)₁: 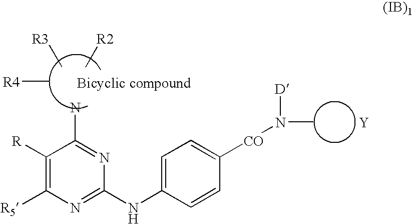
wherein R2, R3, R4, R₅′, D′ and Y are as defined above;
or reacting the compound of formula (XIII) with an amine of formula (VIII)₂ as defined above to obtain a compound of formula (IB)₂: 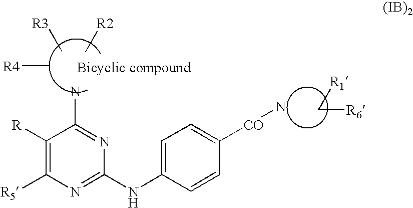
wherein R, R₁′, R2, R3, R4, R₅′ and R₆′ are as defined above;
wherein the compound of formula (IA)1, (IA)2, (IB)1 and (IB)2 can be a compound of formula (I) in which z respectively represents SO₂ or CO; or in order to obtain the compound of formula (I), can optionally be subjected to one or more of the following conversion reactions, in any order:
a) an oxidation reaction on an alkylthio group to give the corresponding sulphoxide or sulphone,
b) a conversion reaction on an alkoxy functional group to give a hydroxyl functional group or also on a hydroxyl functional group to give an alkoxy functional group,
c) an oxidation reaction on an alcohol functional group to give an aldehyde or ketone functional group,
d) an elimination reaction on the protective groups which can be carried by the protected reactive functional groups,
e) a salification reaction by an inorganic or organic acid in order to obtain the corresponding salt, and
f) a resolution reaction on the racemic forms to give resolved products.

30) A method for preparing a compound of formula (IA) as defined in claim 6, wherein Y represents NR10 and wherein R10 represents CH₂—RZ and RZ represents an alkyl, alkenyl or alkynyl radical, which radicals are optionally substituted by a naphthyl radical or by one or more identical or different radicals chosen from halogen, phenyl and heteroaryl radicals, wherein the naphthyl, phenyl and heteroaryl radicals are optionally substituted by one or more identical or different radicals chosen from halogen, hydroxyl, alkoxy, alkyl, hydroxyalkyl, alkoxyalkyl, CF₃, NH₂, NHalk and N(alk)₂ radicals; said method comprising subjecting a compound of formula (XIV) wherein R, R2, R3, R4, and D are as defined in claim 6, and R₅′ and D′ are as defined in claim 6 for R5 and D, respectively, wherein the optional reactive functional groups are optionally protected by protective groups, and z represents SO₂ or CO: 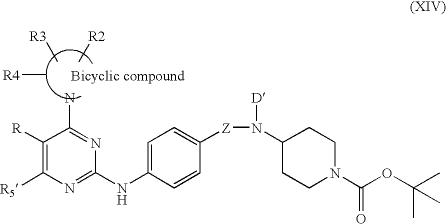
to a reaction for deprotecting the carbamate functional group in order to obtain a compound of formula (XV): 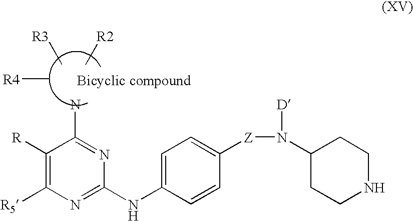
wherein R, R2, R3, R4, R5, z and D′ are as defined above; subjecting the compound of formula (XV) to reducing amination conditions in the presence of an aldehyde or ketone of formula (XVI):
RZ′—CR8′O (XVI)
wherein RZ′ is as defined for Z above and R8′ is as defined in claim 6 for R8, wherein the optional reactive functional groups of Z or R8 are optionally protected by protective groups,
to obtain a compound of formula (IA): 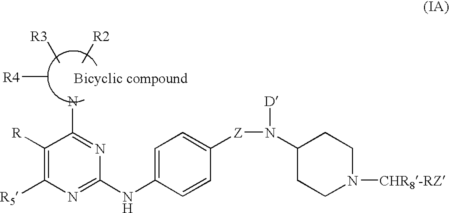
wherein R, R2, R3, R4, R₅′, z, D′, R₈′ and RZ′ are as defined above;
wherein the compounds of formula (IA) can be a compound of formula (I); or in order to obtain a compound formula (I), can be optionally be subjected to one or more of the following conversion reactions, in any order:
a) an oxidation reaction on an alkylthio group to give the corresponding sulphoxide or sulphone,
b) a conversion reaction on an alkoxy functional group to give a hydroxyl functional group or also on a hydroxyl functional group to give an alkoxy functional group,
c) an oxidation reaction on an alcohol functional group to give an aldehyde or ketone functional group,
d) an elimination reaction on the protective groups which can be carried by the protected reactive functional groups,
e) a salification reaction by an inorganic or organic acid in order to obtain the corresponding salt, and
f) a resolution reaction on the racemic forms to give resolved products.

31) A pharmaceutical composition comprising a compound of formula (I) according to claim 1 or a pharmaceutically acceptable salt thereof and a pharmaceutically acceptable vehicle.

32) A pharmaceutical composition comprising a compound formula (I) according to claim 28 or a pharmaceutically acceptable salt thereof and a pharmaceutically acceptable vehicle.

33) A method of inhibiting the activity of the protein kinase IKK comprising contacting said protein kinase with a compound according to claim 1 or a pharmaceutically acceptable salt thereof.

34) The method according to claim 33, wherein the protein kinase is in a mammal.

35) A method of treating or preventing a disease selected from inflammatory diseases, diabetes and cancer, comprising administering to a patient in need of said treatment or prevention a therapeutically effective amount of compound according to claim 1 or a pharmaceutically acceptable salt thereof.

36) The method according to claim 35 wherein the disease is cancer.

37) The method according to claim 36 wherein the cancer is resistant to cytotoxic agents.
